SEQUENCE LISTING (12 sequences)

SEQ ID NO: 1 - protein, 443 aa, Mus musculus
EVHLVESGGGLVKPGGSLKLSCAASGFTFSSYYMYWVRQTPEKRLEWVATISNGGIYTYYLDSVRGRFTISRDNAKNILYLQMSGLSSADSAIYYCTRDGERQGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 215 aa, Mus musculus
QIVLTQSPAIMSVSLGERVTMTCIVSSSVSSTYLHWYQQKPGSSPKLWIYSSSNLASGVPTRFSGSGSGTSHSLTISSMEAEDAAAYYCQLYRRSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 7 aa, Mus musculus
GFTFSSY

SEQ ID NO: 4 - protein, 6 aa, Mus musculus
SNGGIY

SEQ ID NO: 5 - protein, 10 aa, Mus musculus
DGERQGAMDY

SEQ ID NO: 6 - protein, 12 aa, Mus musculus
IVSSSVSSTYLH

SEQ ID NO: 7 - protein, 7 aa, Mus musculus
SSSNLAS

SEQ ID NO: 8 - protein, 9 aa, Mus musculus
QLYRRSPLT

SEQ ID NO: 9 - protein, 119 aa, Mus musculus
EVHLVESGGGLVKPGGSLKLSCAASGFTFSSYYMYWVRQTPEKRLEWVATISNGGIYTYYLDSVRGRFTISRDNAKNILYLQMSGLSSADSAIYYCTRDGERQGAMDYWGQGTSVTVSS

SEQ ID NO: 10 - protein, 108 aa, Mus musculus
QIVLTQSPAIMSVSLGERVTMTCIVSSSVSSTYLHWYQQKPGSSPKLWIYSSSNLASGVPTRFSGSGSGTSHSLTISSMEAEDAAAYYCQLYRRSPLTFGAGTKLELK

SEQ ID NO: 11 - protein, 220 aa, Mus musculus
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCHQHYSTPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 219 aa, Mus musculus
QAQIQLVQSGPELKKPGETVKISCKASGYTFTNYGMNWVKQAPGKGLKWMGWINTYTGEPTYADDFKGRFAFSLETSASTAYLQINNLKNEDMATYFCARGGYYYGNNYEAYWGQGTLVTVSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKI

(19) United States
(12) Patent Application Publication
(10) Pub. No.: US 2025/0163181 A1
(43) Pub. Date: May 22, 2025

(54) ANTI-PROTHROMBIN ANTIBODIES TARGETING THE OPEN FORM OF PROTHROMBIN

(71) Applicants: Saint Louis University, St. Louis, MO (US); Beth Israel Deaconess Medical Center, Inc., Boston, MA (US)

(72) Inventors: Nicola Pozzi, St. Louis, MO (US); Robert Flaumenhaft, Newton, MA (US)

(21) Appl. No.: 18/949,697

(22) Filed: Nov. 15, 2024

Related U.S. Application Data
(60) Provisional application No. 63/599,940, filed on Nov. 16, 2023.

(51) Int. Cl. C07K 16/36; A61K 39/00; A61P 7/02

(52) U.S. Cl. CPC C07K 16/36; A61P 7/02; A61K 2039/505; A61K 2039/54; C07K 2317/55; C07K 2317/565; C07K 2317/92

(57) ABSTRACT

Antigen-binding proteins including monoclonal antibodies (mAbs) against prothrombin, which bind to the open form of prothrombin and decrease conversion of prothrombin to thrombin are provided. Antibody fragments and conjugates are also provided. Anticoagulant pharmaceutical compositions based on the monoclonal antibodies against prothrombin and fragments and conjugates thereof are also provided, along with methods for treating and preventing blood clots and for reducing thrombin generation in patients.

### CROSS REFERENCE TO RELATED APPLICATIONS

This application claims priority to U.S. Provisional Application No. 63/599,940, filed on Nov. 16, 2023, the content of which is incorporated herein by reference in its entirety.

### GOVERNMENT LICENSE RIGHTS

This invention was made with government support under HL150146 and R35HL135775 awarded by National Heart, Lung, and Blood Institute. The government has certain rights in this invention.

### THE NAMES OF PARTIES TO A JOINT RESEARCH AGREEMENT

Not applicable.

### INCORPORATION-BY-REFERENCE OF MATERIAL SUBMITTED ON A READ-ONLY OPTICAL DISC, AS A TEXT FILE OR AN XML FILE VIA THE PATENT ELECTRONIC SYSTEM

The instant application contains a Sequence Listing XML which has been submitted in XML format via EFS-Web and is hereby incorporated by reference in its entirety. Said XML copy, created on Nov. 15, 2025 is named 24004.US SEQ Listing.xml and is 12.4 kilobytes in size.

### FIELD OF THE INVENTION

The invention relates to antigen-binding proteins including monoclonal antibodies against prothrombin and fragments and conjugates thereof, which bind to the open form of prothrombin and decrease conversion of prothrombin to thrombin. The invention is also directed to anticoagulant compositions and methods for treating and preventing blood clots and for reducing thrombin generation in patients.

### BACKGROUND OF THE INVENTION

Coagulation factor II, or prothrombin, is a 72 kDa multi-domain glycoprotein that is important for life. Prothrombin (aa. 1-579) is composed of fragment 1 (aa. 1-155) containing the Gla domain and kringle-1 and fragment 2 (aa. 156-579) containing kringle-2 and the protease domain. The protease domain contains two polypeptide chains, A and B chains, held together by a disulfide bond. Prothrombin has three linkers connecting the N-terminal Gla domain to kringle-1 (Lnk1), the two kringles (Lnk2), and kringle-2 to the C-terminal protease domain (Lnk3)

In plasma, prothrombin is mostly inactive (i.e., zymogen) and circulates in two forms at equilibrium, “closed” (˜80%) and “open” (˜20%), brokered by the flexibility of the linker regions (Pozzi N et al., J Biol Chem. 2016 Aug. 26; 291(35):18107-16; Chinnaraj M et al., Sci Rep. 2018 Feb. 13; 8(1):2945). Its structure remained elusive until 2018 when the first X-ray crystal structure of prothrombin locked in the predominant closed form was published (Chinnaraj M et al., Sci Rep. 2018 Feb. 13; 8(1):2945; Chinnaraj et al., Frontiers in Medicine, Vol. 5:281, 2018).

In response to vascular injury, prothrombin is converted to its active form thrombin by prothrombinase, a macromolecular complex composed of factor Xa (fXa), factor Va (fVa), calcium ions, and phospholipids. The prothrombinase complex generates thrombin by cleaving prothrombin at Arg-271 and Arg-320 (Chinnaraj et al., Frontiers in Medicine, Vol. 5:281, 2018). Once in the circulation, thrombin converts fibrinogen into fibrin, activates platelets, sustains immune response and increases endothelial permeability thereby halting the loss of blood at the site of injury and facilitating vascular remodeling. Excess thrombin results in blood clots. Thus, prothrombin and thrombin are targets of anticoagulant therapy.

Thrombosis (i.e., excess blood clotting) is a serious and often fatal disease in which blood clots (thrombi) interfere with the normal flow of blood in blood vessels or the heart. Fibrin is a protein generated from the action of the blood coagulation protein thrombin on fibrinogen. A thrombus is a deposit of blood components, primarily fibrin with red blood cells or aggregated platelets on the lining or surface of a blood vessel or cavity of the heart. A thrombus includes insoluble fibrin polymers that are later decomposed through fibrinolysis. Thrombi can obstruct normal blood flow, leading to serious and often fatal consequences.

A number of disorders and diseases exhibit an increased tendency to thrombosis. These include, but are not limited to cardiovascular disease, cancer, pregnancy, aging, trauma, oral contraceptive use, diabetes mellitus, liver and kidney disease, COVID, and obesity. Furthermore, antiphospholipid syndrome (APS) is an autoimmune disorder characterized by vascular thromboses in the presence of antiphospholipid antibodies. A prominent target of antiphospholipid antibodies is the clotting factor prothrombin.

There are several anticoagulants on the market, such as heparin and warfarin; however, there is a need for additional and/or more effective anticoagulants. For example, heparin binds to a number of plasma proteins in the bloodstream, which reduces its anticoagulant activity at low concentrations, thereby contributing to the variability of the anticoagulant response among patients with thromboembolic disorders and to the laboratory phenomenon of heparin resistance. Heparin also binds to endothelial cells and macrophages, properties that further complicate its pharmacokinetics and to von Willebrand factor, which inhibits von Willebrand factor-dependent platelet function. Newer anticoagulants, novel oral anticoagulants (NOAC) or directly acting oral anticoagulants (DOAC) include a direct thrombin inhibitor (dabigatran) and factor Xa inhibitors (rivaroxaban, apixaban, and edoxaban). These medications work differently than warfarin while achieving similar anticoagulation effects and exhibiting similar side effects. Furthermore, they are approved for a subset of conditions and cannot be broadly used as anticoagulants.

### SUMMARY OF THE INVENTION

The invention provides antigen-binding proteins against prothrombin. These antigen-binding proteins, which include monoclonal antibodies bind to the open form of prothrombin and decrease conversion of prothrombin to thrombin.

The invention further provides antigen-binding proteins that bind to fragment 1 and Linker 2 of prothrombin and decrease conversion of prothrombin to thrombin.

Also provided are antigen-binding proteins, which bind to kringle-1 of prothrombin and decrease conversion of prothrombin to thrombin.

The invention is further directed to antigen-binding proteins, which bind to at least one of the following, or alternatively to at least two of the following residues E85, L88, S91, Y93, R90, R92, Q110, and E111 in the kringle-1 domain of prothrombin.

The antigen-binding proteins can include monoclonal antibodies and fragments thereof that have a dissociation constant (Kd) of less than about 100 nM.

The monoclonal antibodies can be chimeric, humanized, human or murine.

The invention also provides fragments of the monoclonal antibodies including Fab fragments, (Fab′)2 fragments, single chain antibody fragments (scFv), variable domains derived from heavy chain antibodies (hcAb) called VHHs and conjugates of monoclonal antibodies and fragments thereof.

The invention is also directed to anticoagulant compositions comprising any of the monoclonal antibodies or fragments or conjugates thereof.

Methods that use the anticoagulant compositions for treatment and prevention of blood clots and for reducing thrombin generation in a patient are also provided. The compositions can be used to treat patients who have or are at risk for deep vein thrombosis (DVT), pulmonary embolism (PE), atrial fibrillation (AFib), myocardial infarction (MI), ischemic stroke, unstable angina, hip or knee replacement surgery, COVID, antiphospholipid syndrome (APS), systemic lupus erythematosus (SLE), or cancer-associated thrombosis (CAT).

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1, panel A depicts human prothrombin (Uniprot entry: P00734), which is made of 4 domains that are connected by three linkers. Fragment-1, comprising residues 1-155, was selected for immunization experiments.

FIG. 1, panel B shows conformational equilibrium of prothrombin showing the relocation of fragment-1 (red) relative to the protease domain (yellow) and location of residues S91 and Y93 in kringle-1.

FIG. 1, panel C depicts ELISA experiments with POmAb. POmAb was incubated with specified proteins, and binding was detected with an anti-mouse gamma-specific IgG antibody conjugated with HRP and TMB substrate. Each binding experiment was independently repeated a minimum of three times.

FIG. 1, panels D, E, F, G and H show surface plasmon resonance experiments, with reference-subtracted real-time association and dissociation profiles resulting from flowing kringle-1 (0-333 nM) (FIG. 1, panel D), kringle-2 (0-333 nM) (FIG. 1, panel E), prothrombin (ProT) (0-1000 nM) (FIG. 1, panel F), prothrombin (0-1000 nM) with 200 μM argatroban (FIG. 1, panel G) and prothrombin variant S91A/Y93A (0-2000 nM) (FIG. 1, panel H) onto immobilized POmAb. Rate constants are reported in Table 1. FIG. 1, panel I shows FRET histograms of prothrombin labeled at residues 101 and 478 in the absence (top, yellow) and presence of 500 nM POmAb (bottom, purple).

FIG. 2, panels A-D depict cryo-EM structure of POmAb bound to prothrombin and activity in human plasma. FIG. 2, panel A shows the Fab fragment of POmAb comprising heavy (purple, HC and VH) and light (violet, LC and VL) chains bound to kringle-1 (cyan). FIG. 2, panel B depicts interacting surfaces of kringle-1 (top) and Fab (bottom) colored according to the electrostatic potential (blue=electropositive, red=electronegative). Residue R90 in kringle-1 is attracted by residue D314 of the antibody, resulting in electrostatic complementarity. Hydrophobic/hydrophilic interactions mediated by Y93 in kringle-1 and a cluster of tyrosine residues (Y92, Y248, Y250, and Y274) and in the antibody provide additional binding energy. FIG. 2, panel C is a zoomed-in characterization of the binding interface between kringle-1 (cyan) and POmAb (purple), with energetically favorable interactions shown between the two proteins. Antibody residues are shown in wheat. FIG. 2, panel D shows the crystal structure of the closed form of prothrombin solved at 4.1 Å resolution (6CW2.pdb, yellow) overlayed on the cryo-EM structure of the POmAb/kringle-1 complex showing how the protease domain clashes against the antibody, explaining why POmAb cannot bind the closed form. In the closed form, residue S91 of kringle-1 interacts with residue D552 of the protease domain, while residue Y93 of kringle-1 interacts with residues W468 and W547 of the protease domain. Binding of the protease domain and POmAb to residues R90-Y93 of kringle-1 is, therefore, mutually exclusive.

FIG. 3, panels A-C depict an anticoagulant effect of POmAb in human plasma. aPTT is shown in FIG. 3, panel A and dRVV times are shown in FIG. 3, panels B and C of human plasma measured at increasing concentrations of POmAb (0-5 μM). dRVV time was measured at low (FIG. 3, panel B) and high (FIG. 3, panel C) phospholipids content. FIG. 3, panel D shows clotting time measured in prothrombin-deficient plasma supplemented with prothrombin wild-type or the variant S91A/Y93A.

FIG. 4 depicts the annotated amino acid sequence of POmAb, with light chain on top and heavy chain on the bottom. Variable regions are colored in black, and constant regions are colored in blue. Complementarity determining regions (CDRs) are colored in orange.

FIG. 5, panels A-D depict cryo-EM structure of POmAb in complex with kringle-1. FIG. 5, panel A depicts the overview of cryo-EM data processing strategy through cryoSPARC. FIG. 5, panel B depicts Fourier shell correlation curves of unmasked and masked map. Resolution was calculated using two half-maps and a gold-standard Fourier shell correlation coefficient of 0.143 (blue dashed line). FIG. 5, panel C depicts sharpened density map (contour level 0.9) of the complex, and FIG. 5, panel D depicts sharpened density map of the complex colored by local resolution in Angstroms (red=higher resolution; blue=lower resolution).

FIG. 6, panels A-B show post-processing and refinement statistics of the prothrombin and POmAb Fab fragment complex. Model of the complex fit to the cryo-EM density map (sharp) of the complex post-processed with DeepEMhancer in cryoSPARC is shown in FIG. 6, panel A. Map counter level is 0.1. FIG. 6, panel B shows Phenix Real space refinement statistics of the reconstructed model. Note how there is additional electron density protruding from kringle-1, consistent with the presence of Lnk1 (residues 40-61) and the Gla-domain (residues 1-39). Modeling this part of prothrombin was not attempted for two reasons. Firstly, the resolution of the map is low in this region, making modeling efforts very error-prone. Second, the model indicates that this region is not part of the binding interface. This conclusion is confirmed by binding studies in FIG. 1, showing that POmAb specifically interacts with kringle-1 and not does not require any other part of prothrombin.

FIG. 7 depicts reactivity of 5 selected clones towards prothrombin, prothrombin fragments, and variant S91A/Y93A tested by ELISA. POmAb reacts with prothrombin (ProT), gla-domainless prothrombin (GD-ProT), fragment-1, and kringle-1 but fails to react against kringle-2 and the variant S91A/Y93A (p<0.001, ***). Unlike POmAb, clones 2F10, 5B10, 12H12, and 15E9 did react well with the variant S91A/Y93A (ns), indicating their epitope is in kringle-1 but distinct from that of POmAb.

FIG. 8, panels A-D depict antibody-stimulated binding of prothrombin to liposomes as monitored by SPR. Sensograms of prothrombin wild-type (WT) are shown in FIG. 8, panels A and B and of variant S91A/Y93A in FIG. 8, panels C and D, in the absence (black lines) and presence (purple lines) of 2.5 μg/ml of POmAb IgG (FIG. 8, panels A and C) and Fab fragment (FIG. 8, panels B and D). The binding of prothrombin wilt-type is stimulated only by intact IgG, which is bivalent, but not by Fab, which is monovalent. Also, a minimal effect is seen with the variant S91A/Y93A because of the much lower affinity for POmAb compared to prothrombin wild-type.

FIG. 9, panels A-E depict the anticoagulant effect that POmAb exerts in human plasma by forcing prothrombin to open. FIG. 9, panel A shows antibody-stimulated binding of prothrombin wild-type (250 nM) and variant S91A/Y93A (250 nM) to negatively charged liposomes (POPC:POPS 80:20) monitored by SPR. IgG (gray) and Fab (green) POmAb were used at 2.5 μg/ml. Values of fold increase were determined using the formula RUlgG/Fab/RUfree at 115 sec. The horizontal dotted line set at y=1.0 indicates there is no effect of the treatment. Raw data are shown in FIG. 8. In FIG. 9, panel B, aPTT of human plasma was measured at increasing concentrations of POmAb IgG and Fab (0-5 UM). FIG. 9, panel C shows dRVV time expressed as normalized LA-ratio as per manufacturer's instructions. Shown are normal control plasma (−ve CTRL), LA-control plasma (+ve CTRL), and normal control plasma supplemented with 1 μM POmAb. The horizontal dotted line set at y=1.2 indicates the threshold provided by the manufacturer for considering a specimen LA positive (above threshold) or negative (below threshold). FIG. 9, panel D depicts clotting time as measured in prothrombin-deficient plasma supplemented with prothrombin wild-type or the variant S91A/Y93A. FIG. 9, panel E depicts continuous assay monitoring of the conversion of prothrombin to thrombin by prothrombinase complex. The assay started with the addition of 2.5 pM factor Xa to 25 nM prothrombin that was preincubated for 20 minutes with 20 μM phospholipids, 2 nM cofactor Va, and increasing concentrations of Fab (0-2 μM). The release of p-nitroaniline upon hydrolysis of FPF was monitored at 405 nm. FIG. 9, panel F depicts SDS-PAGE analysis of the conversion of prothrombin (1.2 M) to thrombin by the prothrombinase complex (0.2 nM factor Xa, 20 μM phospholipids, 10 nM Va) in the absence and presence of 1.5 μM Fab POmAb, which shows as the two bands at 25 kDa because of the presence of reducing agent. The disappearance of prothrombin (PT, 72 kDa band) is slower in the presence of Fab and is linked to lower accumulation of B-chain (28 kDa) and A-chain (6 kDa), which are signatures of thrombin. Moreover, in the presence of Fab, there is significantly more accumulation of a band at 50 kDa (black arrow), compatible with cleavage at R155 and the formation of prethrombin-1. FIG. 9, panel G shows the proposed mechanism of action of POmAb resulting in anticoagulation. In the blood, prothrombin mostly circulates in closed form, and upon binding to phospholipids, does not spontaneously open as suggested by SPR data in FIG. 9, panel A. It requires POmAb to shift the equilibrium. PTC=prothrombinase complex. *** p<0.001, ** p<0.01, n.s. not significant.

FIG. 10, panels A-E depict the characterization of the monoclonal anti-kringle-1 antibody 5B10 and comparison with POmAb. FIG. 10, panel A shows the reactivity of POmAb and FIG. 10, panel B shows the reactivity of 5B10 towards prothrombin, kringle-1, kringle-2, and variant S91A/Y93A monitored by ELISA. POmAb and 5B10 reacted with similar affinity toward immobilized prothrombin and kringle-1 but failed to react against kringle-2. However, unlike POmAb, 5B10 reacted well with the variant S91A/Y93A, indicating its epitope is in kringle-1 but distinct from that of POmAb. Values of EC₅₀ determined by ELISA are reported in Table 5. FIG. 10, panel C shows smFRET experiments of prothrombin 101-478 (80 pM) unbound and bound to POmAb (500 nM, purple) and 5B10 (500 nM, blue). Like POmAb, 5B10 shifted the conformational equilibrium towards the open form of prothrombin, which was characterized by a low FRET signal. Experimental conditions were 20 mM Tris-HCl PH 7.4, 150 mM NaCl, 5 mM CaCl₂) and 0.01% Tween 20. FIG. 10, panel D shows aPTT of human plasma measured at 1 μM POmAb (purple) and 1 μM 5B10 (blue). FIG. 10, panel E depicts dRVV time expressed as normalized LA-ratio as per manufacturer's instructions. Shown are normal control plasma (−ve CTRL), LA-control plasma (+ve CTRL), and normal control plasma supplemented with POmAb (1 μM) and 5B10 (1 μM). The horizontal dotted line set at y=1.2 indicates the threshold provided by the manufacturer for considering a specimen LA positive (above threshold) or negative (below threshold). *** p<0.001, ** p<0.01, n.s. not significant.

FIG. 11, panels A-F depict characterization of the monoclonal anti-kringle-2 antibody AHP and comparison with POmAb. FIG. 11, panels A and B depict reactivity of POmAb and AHP, respectively, towards prothrombin, kringle-1, kringle-2, and variant S91A/Y93A monitored by ELISA. Like POmAb, AHP reacted with immobilized prothrombin, albeit with lower affinity. In contrast to POmAb, AHP did not react with kringle-1 but instead reacted with kringle-2 and was insensitive to the mutations S91A and Y93A. This shows that AHP is an anti-kringle-2 antibody. Values of EC₅₀ determined by ELISA are reported in Table 5. FIG. 11, panels C and D are SPR experiments reporting the binding of immobilized AHP to prothrombin in the absence and presence of 200 μM argatroban, respectively. Unlike POmAb, AHP reacted with closed and open forms of prothrombin with similar affinity, indicating the epitope is equally exposed in both conformations. Values of on and off rates for prothrombin, prothrombin with argatroban, kringle-1, kringle-2, and variant S91A/Y93A are reported in Table 6. FIG. 11, panel E shows smFRET experiments of prothrombin 101-478 (80 pM) unbound and bound to POmAb (500 nM, purple) and AHP (500 nM, gray). Unlike POmAb, AHP does modify the conformational equilibrium, suggesting it binds to both closed and open forms. Experimental conditions were 20 mM Tris-HCl PH 7.4, 150 mM NaCl, 5 mM CaCl₂) and 0.01% Tween 20. FIG. 11, panel F shows dRVV time expressed as normalized LA-ratio as per manufacturer's instructions. Shown are normal control plasma (−ve CTRL), LA-control plasma (+ve CTRL), and normal control plasma supplemented with POmAb (1 μM) and AHP (500 nM). The horizontal dotted line set at y=1.2 indicates the threshold provided by the manufacturer for considering a specimen LA positive (above threshold) or negative (below threshold). Unlike POmAb, AHP produces a profound LA effect. *** p<0.001, ** p<0.01, n.s. not significant.

FIG. 12, panels A-G depict the effect of type I and type II antibodies on thrombin generation and activated protein C resistance. FIG. 12, panel A shows the subtype and mechanism of action of monoclonal antiprothrombin antibodies POmAb, 5B10, and AHP. FIG. 12, panels B-D depict thrombin generation experiments obtained by adding 20 μL of buffer (0) or 50, 100, and 150 μg/mL of POmAb, 5B10, and AHP, respectively, in the absence of 10 nM activated protein C (aPC), and FIG. 12, panels E-G show the same experiments in the presence of 10 nM aPC. Final antibody concentrations of the antibodies in the assay (120 μL) are 0, 8, 16, and 32 μg/mL.

FIG. 13 depicts the annotated amino acid sequence of 5B10 monoclonal antibody, with light chain on top and heavy chain on the bottom. Variable regions are colored in black, and constant regions are colored in blue. Complementarity determining regions (CDRs) are highlighted in yellow.

FIG. 14, panels A-C depict the ability of POmAb to reduce fibrin formation in vivo, acting as an anticoagulant. FIG. 14, panel A shows the kinetics of fibrin formation in animals treated with control antibody (black, CTRL) or POmAb at 0.5 mg/Kg (blue). FIG. 14, panel B shows quantification of platelets (red) and fibrin (green), revealing statistically lower fibrin signals in treated animals. FIG. 14, panel C shows images at 0, 30, 60, 90, 120, and 240 seconds documenting accumulation of POmAb (blue) at the injury site, followed by fibrin (green) and platelets (red).

### DEFINITIONS

When introducing elements of the invention, the articles “a”, “an”, “the” and “said” are intended to mean that there are one or more of the elements. Thus, for example, reference to “a viral antigen” includes a multitude of such antigens, and so forth.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to a conformational epitope of prothrombin in open form.

The term “antigen-binding protein,” “binding protein” or “binding molecule,” as used herein includes molecules that contain at least one antigen-binding site that specifically binds to kringle-1 domain of prothrombin in the open form. A binding protein may be an antibody, such as a full-length antibody, or an antigen-binding fragment of an antibody or a single chain antibody fragment, or a conjugate of any of the above.

The terms “comprising”, “including” and “having” are intended to be inclusive and mean that there may be additional elements other than the listed elements.

By the terms “effective amount” and “therapeutically effective amount” of a composition or composition component is meant a sufficient amount of the composition or component, alone or in a combination, to provide the desired effect, such as prevention of disease or inhibition of one or more of the symptoms of disease, for which the composition or composition component is being administered.

The term “epitope” refers to an antigenic determinant that interacts with a specific antigen-binding site in the variable region of an antigen-binding protein known as a paratope. A single antigen may have more than one epitope. Thus, different antigen-binding proteins may bind to different areas on an antigen and may have different biological effects. Epitopes may also be “conformational,” that is, composed of non-linear amino acids.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pies, horses, goats, and the like), etc.

The term “human antibody”, as used herein, is intended to include antibodies having variable and constant regions derived from human germline immunoglobulin sequences.

The term “Kd”, as used herein, is intended to refer to the equilibrium dissociation constant of a particular antigen-binding protein-antigen interaction.

“Monoclonal antibodies” refer to antibodies produced from a single B cell or B cell line such that all of monoclonal antibodies originating from said B cell or B cell line bind the same antigen epitope(s).

The term “recombinant”, as used herein, refers to antigen-binding proteins, e.g., antibodies or antigen-binding fragments thereof, of the invention created, expressed, isolated or obtained by technologies or methods known in the art as recombinant DNA technology which include, e.g., DNA splicing and transgenic expression. The term refers to antigen-binding proteins, e.g., antibodies expressed in a non-human mammal (including transgenic non-human mammals, e.g., transgenic mice), or a cell (e.g., CHO cells) expression system or isolated from a recombinant combinatorial human antibody library.

The term “specifically binds,” or “binds specifically to”, or the like, means that an antigen-binding protein, e.g., antibody, or antigen-binding fragments thereof, forms a complex with an antigen that is relatively stable under physiologic conditions. Specific binding can be characterized by an equilibrium dissociation constant of at least about 1×10-8 M or less (e.g., a smaller Kd denotes a tighter binding). Methods for determining whether two molecules specifically bind are well known in the art and include, for example, equilibrium dialysis, surface plasmon resonance, and the like. As described herein, antigen-binding proteins, e.g., antibodies, have been identified by surface plasmon resonance.

The term “substantial identity” or “substantially identical,” when referring to an amino acid sequence or fragment thereof, indicates that, when optimally aligned with appropriate amino acid insertions or deletions with another polypeptide, there is the sequence identity in at least about 90%, and more preferably at least about 95%, 96%, 97%, 98% or 99% of the amino acids, as measured by any well-known algorithm of sequence identity, as discussed below.

The terms “treatment”, “treating” and the like are used herein to generally mean obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease or in delaying the disease. “Treatment” or “treating” or the like as used herein covers any treatment of a disease or symptom in a mammal, and includes: (a) preventing the disease or symptom from occurring in a subject who may be predisposed to acquiring the disease or symptom but has not yet been diagnosed as having it; (b) inhibiting the disease or symptom, i.e., arresting its development; or (c) relieving the disease, i.e., causing regression of the disease. Inhibiting or preventing a symptom means that an improvement is observed in the subject with respect to symptoms associated with the underlying disease, notwithstanding that the subject may still be afflicted with the underlying disease.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable sub-combination. All combinations of the embodiments pertaining to the invention are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### DESCRIPTION OF THE PREFERRED EMBODIMENTS

As has been determined recently, prothrombin exists in two forms at equilibrium, closed (˜80%) and open (˜20%) (Pozzi N et al., J Biol Chem. 2016 Aug. 26; 291(35):18107-16; Chinnaraj M et al., Sci Rep. 2018 Feb. 13; 8(1):2945; Chinnaraj et al., Frontiers in Medicine, Vol. 5:281, 2018). Prothrombin transitions from an “L-closed” to an “I-open” shape due to the relocation of fragment-1 away from the protease domain. In the closed form, kringle-1 sits on top of the catalytic pocket, blocking access of substrates to the active site, as well as hiding portions of the flexible autolysis loop (Chinnaraj et al., Frontiers in Medicine, Vol. 5:281, 2018).

Antigen-binding proteins have been discovered that include monoclonal antibodies (mAbs) against prothrombin, which bind to the open form of prothrombin and decrease conversion of prothrombin to thrombin.

The antigen-binding proteins against prothrombin can decrease conversion of prothrombin to thrombin and bind to Fragment 1 and Linker 2 (Lnk2), which connects kringle-1 and kringle-2.

The antigen binding proteins of the invention, and particularly monoclonal antibodies, can bind to kringle-1 of prothrombin and decrease conversion of prothrombin to thrombin.

While not being bound to a particular theory, it is believed that by interacting with kringle-1, the antigen-binding proteins of the invention clash against the protease domain, forcing prothrombin to open.

The antigen-binding proteins can bind to the kringle-1 stretch of amino acids including but not limited to glutamate at position 85, leucine at position 88, arginine at position 90, serine at position 91, arginine at position 92, tyrosine at position 93, glutamine at position 110 or glutamate at position 111. In some instances, the antigen-binding proteins bind to at least one of the following residues E85, L88, R90, S91, R92, Y93, Q110, E111 in the kringle-1 domain of the open form prothrombin. In other instances, the antigen-binding proteins bind to at least two of the following residues E85, L88, R90, S91, R92, Y93, Q110, E111 in the kringle-1 domain. The at least two residues can be, for example, serine at position 91 (S91) and tyrosine at position 93 (Y93).

The antigen-binding proteins of the invention can react strongly against prothrombin, gla-domainless prothrombin, and kringle-1 but show a significantly reduced binding activity for the prothrombin variant, whose kringle-1 region has been mutated such that residues described above have been mutated to alanine. For example, in the experiments described in the Examples, the prothrombin variant has serine at position 91 and tyrosine at position 93 mutated to alanines (S91A and Y93A).

In some instances, the antigen-binding proteins of the invention do not bind kringle-2 and other plasma proteins containing kringle domains like plasminogen, tissue plasminogen activator, and factor XII, as well as other antigens of antiphospholipid antibodies like β2-glycoprotein I.

The invention is directed to a monoclonal antibody against prothrombin, which binds the open form of prothrombin and decreases conversion of prothrombin to thrombin.

The invention is directed to monoclonal antibodies, which bind to Fragment 1 and Linker 2 of prothrombin and it is furthermore directed to monoclonal antibodies, which bind to kringle-1 of prothrombin in the open form. Regardless of the specific open form prothrombin epitopes with which they interact, the monoclonal antibodies of the invention decrease conversion of prothrombin to thrombin.

The monoclonal antibody can be a human antibody. The monoclonal antibody can also be a murine antibody. In some instances, the mAb can be a humanized antibody. Humanized antibodies are antibodies from non-human species whose protein sequences have been modified to increase their similarity to antibody variants produced naturally in humans. More specifically, a humanized antibody has segments of foreign-derived amino acids interspersed among variable domain segments of human-derived amino acid residues, and the humanized variable heavy and variable light domains are linked to heavy and light constant regions of human origin. For example, antibodies produced in mice are routinely humanized for administration to human patients. Humanized antibodies can be readily generated by one of ordinary skill in the art. In other embodiments, the mAbs can be chimeric. Chimeric antibodies are structural chimeras made by fusing variable regions from one species such as a mouse, with the constant regions from another species such as a human being. In particular, a chimeric antibody is one that contains contiguous foreign-derived amino acids comprising the entire variable domain of both heavy and light chains linked to heavy and light constant regions of human origin. Chimerization of antibodies is done to reduce immunogenicity and increase serum half-life when preparing monoclonal antibodies (mAbs), usually for therapeutic purposes. Similarly to humanized antibodies, chimerization of monoclonal antibodies is routinely done in the art. See, e.g. Dang V T, Mandakhalikar KD, Ng OW, Tan Y J, “A simple methodology for conversion of mouse monoclonal antibody to human-mouse chimeric form,” Clin Dev Immunol, 2013; 2013:716961.

An aspect of the invention is the provision of a mouse monoclonal antibody having the amino acid sequence of the heavy chain as shown in SEQ ID NO: 1 and the amino acid sequence of the light chain as shown in SEQ ID NO: 2 (referred to in the Examples as POmAb). Thus, an aspect of the invention is the provision of a murine monoclonal antibody POmAb.

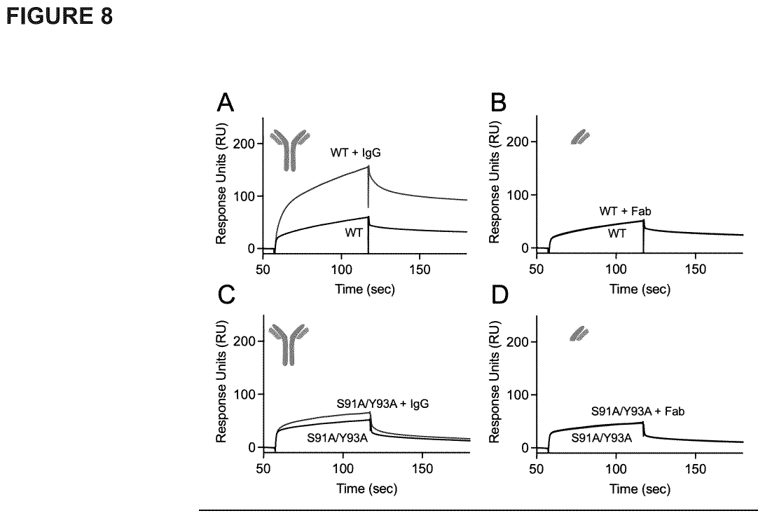

Additional mouse monoclonal antibodies, which bind to prothrombin in the open form and decrease conversion of prothrombin to thrombin include 2F10, 5B10, 15E9, and 12H12, which are disclosed in the Examples and FIG. 8. The heavy and light chain sequences of 5B10 are in SEQ ID Nos: 11 and 12. An aspect of the invention is the provision of a murine monoclonal antibody 5B10.

Another aspect of the invention provides antigen-binding proteins that bind to prothrombin in the open form with high affinity. For example, the invention includes antigen-binding proteins that bind to kringle-1 domain of open prothrombin with a Kd at 25° C. of less than about 100 nM when measured by surface plasmon resonance as described in the Examples or by a substantially similar assay. The Kd can be less than 50 nM, less than 25 nM, less than 20 nM, or less than 10 nM. For example, the Kd of the antigen-binding proteins of the invention can be from about 2 nM to about 20 nM, from about 2 nM to about 10 nM, from about 2 nM to about 8 nM, or from about 2.2 nM to about 5.2 nM.

Antigen-binding proteins can comprise a heavy chain CDR1 (HCDR1) with SEQ ID NO: 3 or a substantially similar sequence thereof having at least 90%, at least 95%, at least 96%, at least 97%, at least 98% or at least 99% sequence identity.

Antigen-binding proteins can comprise a heavy chain CDR2 (HCDR2) comprising an amino acid sequence of SEQ ID NO: 4 or a substantially similar sequence thereof having at least 90%, at least 95%, at least 96%, at least 97%, at least 98% or at least 99% sequence identity.

Antigen-binding proteins can comprise a heavy chain CDR3 (HCDR3) comprising an amino acid sequence of SEQ ID NO: 5 or a substantially similar sequence thereof having at least 90%, at least 95%, at least 96%, at least 97%, at least 98% or at least 99% sequence identity.

Antigen-binding proteins can comprise a light chain CDR1 (LCDR1) comprising an amino acid sequence of SEQ ID NO: 6 or a substantially similar sequence thereof having at least 90%, at least 95%, at least 96%, at least 97%, at least 98% or at least 99% sequence identity.

Antigen-binding proteins can comprise a light chain CDR2 (LCDR2) comprising an amino acid sequence of SEQ ID NO: 7 or a substantially similar sequence thereof having at least 90%, at least 95%, at least 96%, at least 97%, at least 98% or at least 99% sequence identity.

Antigen-binding proteins can comprise a light chain CDR3 (LCDR3) SEQ ID NO: 8 or a substantially similar sequence thereof having at least 90%, at least 95%, at least 96%, at least 97%, at least 98% or at least 99% sequence identity.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antigen-binding protein.

The framework regions (FRs) of the antibody-biding proteins (or antigen-binding fragments thereof) can be identical to the human germline sequences, or can be naturally or artificially modified. An amino acid consensus sequence may be defined based on a side-by-side analysis of two or more CDRs. The human monoclonal antibodies (mAbs) of the invention can include amino acid residues not encoded by human germline immunoglobulin sequences (e.g., mutations introduced by random or site-specific mutagenesis in vitro or by somatic mutation in vivo), for example in the CDRs and in particular CDR3.

Substitution of one or more CDR residues or omission of one or more CDRs is also possible. Antigen binding proteins, such as antibodies, have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

The invention also provides substantially similar antigen-binding proteins to the sequences disclosed herein. Substantially similar antigen-binding proteins include the ones that share at least 90% sequence identity when optimally aligned with the antigen-binding protein sequences disclosed herein, using default gap weights. More preferably, the substantial similarity includes at least 95%, 96%, 97%, 98%, or 99% sequence identity. Additionally, it is preferred that the residue positions, which are not identical, differ by conservative amino acid substitutions. A conservative amino acid substitution is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is incorporated herein by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, incorporated herein by reference. A moderately conservative replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutant thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402.

An antigen-binding protein of the invention can include an antibody fragment. Non-limiting examples of antigen-binding fragments of an antibody include: (i) Fab fragments; (ii) F(ab′)2 fragments; (iii) Fd fragments; (iv) Fv fragments; (v) single-chain Fv (scFv) molecules; (vi) single domain antibodies (dAb fragments); (vii) variable domains derived from heavy chain antibodies (hcAb) called VHHs; and (viii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, domain-deleted antibodies, CDR-grafted antibodies, diabodies, triabodies, tetrabodies, minibodies, and nanobodies (e.g. monovalent nanobodies, bivalent nanobodies, etc.) are also encompassed within antigen-binding fragments. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

The antibody fragment of the invention can be a Fab fragment. An aspect of the invention is the provision of a Fab fragment of POmAb antibody. In other instances, the fragment is the F(ab′)2 fragment, and in still other instances, it is a single chain Fv (scFv) molecule.

An antigen-binding fragment of an antibody can contain at least one variable domain covalently linked to at least one constant domain. Non-limiting, exemplary configurations of variable and constant domains that can be found within an antigen-binding fragment of an antigen-binding protein of the invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains can be either directly linked to one another or can be linked by a full or partial hinge or linker region. A hinge region can consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody can comprise a homo-dimer or heterodimer (or other multimer) of any of the variable and constant domain configurations listed above in non-covalent association with one another and/or with one or more monomeric VH or VL domain (e.g., by disulfide bond(s)).

Methods for generating antigen-binding proteins, such as human antibodies, in transgenic mice are well known in the art. Any such known methods can be used in the context of the invention to make human antibodies that specifically bind to a conformational epitope of prothrombin in the open form. The examples below describe a method that was used to generate monoclonal antibodies in transgenic mice; however, many similar procedures can be used.

By way of example and not of limitation, VELOCIMMUNE® technology (see, for example, U.S. Pat. No. 6,596,541, Regeneron Pharmaceuticals, VELOCIMMUNE®) or any other known method can be used for generating antigen-binding proteins that bind to kringle-1 of prothrombin in the open form and decrease conversion of prothrombin to thrombin. For example, monoclonal antibodies can be initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antigen-binding protein, e.g., antibody, comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNER mouse is challenged with the antigen of interest, such as prothrombin, fragment-1 of prothrombin or kringle-1 region of prothrombin, and lymphatic cells (such as B-cells) are recovered from the mice that express antigen-binding proteins, e.g., antibodies. The lymphatic cells can be fused with a myeloma cell line to prepare immortal hybridoma cell lines, and such hybridoma cell lines can be screened and selected to identify hybridoma cell lines that produce antibodies specific to the antigen of interest. DNA encoding the variable regions of the heavy chain and light chain can be isolated and linked to desirable isotypic constant regions of the heavy chain and light chain. Such an antigen-binding protein can be produced in a cell, such as a CHO cell. Alternatively, DNA encoding the antigen-specific antigen-binding proteins, e.g., chimeric antibodies, or the variable domains of the light and heavy chains can be isolated directly from antigen-specific lymphocytes.

Antigen-binding proteins of the invention encompass proteins having amino acid sequences that vary from those of the described antigen-binding proteins, e.g., the monoclonal antibodies, but that retain the ability to bind a conformational epitope of prothrombin in the open form.

New antigen-binding proteins can readily be generated based on the monoclonal antibody disclosed herein, having the amino acid sequence of SEQ ID NO: 1. One can easily determine whether a newly generated antigen-binding protein binds to the same epitope as, or competes for binding with, a reference anti-kringle-1 antigen-binding protein by using routine methods known in the art. The Examples detail the experiments used to confirm binding of the mAb with SEQ ID NO: 1 to kringle-1 of prothrombin by using ELISA and surface plasmon resonance. In addition to these two techniques, various techniques known to one of ordinary skill in the art can be used to determine whether an antigen-binding protein interacts with one or more amino acids within a polypeptide or protein. Exemplary techniques include, for example, routine cross-blocking assays, such as that described in Antibodies, Harlow and Lane (Cold Spring Harbor Press, Cold Spring Harbor, N.Y.). Other methods include alanine scanning mutational analysis, peptide blot analysis (Reineke (2004) Methods Mol. Biol. 248:443-63), peptide cleavage analysis crystallographic studies and NMR analysis. In addition, methods such as epitope excision, epitope extraction and chemical modification of antigens can be employed (Tomer (2000) Prot. Sci. 9:487-496).

Another method that can be used to identify the amino acids within a polypeptide with which an antigen-binding protein interacts is hydrogen/deuterium exchange detected by mass spectrometry. In general terms, the hydrogen/deuterium exchange method involves measuring the rate at which hydrogens exchange to deuterium in the protein of interest. This is done in the presence and absence of the antigen-binding protein to identify the areas of the protein that are in contact with it. The areas that are in contact with the antigen-binding protein are expected to exchange more slowly, while areas that are not in contact with it will not be affected. To analyze the results, the reactions are quenched by acid pH, proteins are cleaved by proteases, and the protein fragments are then analyzed by mass spectrometry. Protected areas that are in contact with the antigen-binding protein will show lower mass when compared to when the antigen-binding protein is not present. In contrast, areas that are insensitive to binding will have the same rate of exchange. This information is then used to define the specific amino acids with which the antigen-binding protein interacts. See, e.g., Ehring (1999) Analytical Biochemistry 267:252-259; Engen and Smith (2001) Anal. Chem. 73: 256A-265A.

The invention is also directed to conjugates of any of the antigen-binding proteins. Accordingly, any of the monoclonal antibodies, whether they are humanized, chimeric, human, or murine, or any of the antibody fragments can be conjugated to another molecule, such as a label. The label can be, for example, a biotin, an enzyme reporter such as horseradish peroxidase (HRP), alkaline phosphatase (AP), glucose oxidase or β-galactosidase, or a fluorescent tag such as fluorescein, cyanine and rhodamine dye. POmAb, 2F10, 5B10, 15E9 or 12H12 can be coupled to a label.

The invention further provides bioequivalent antigen-binding proteins. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antigen-binding proteins or antibodies will be considered equivalents or pharmaceutical alternatives if they are equivalent in the extent of their absorption but not in their rate of absorption and yet can be considered bioequivalent because such differences in the rate of absorption are intentional and are reflected in the labeling, are not essential to the attainment of effective body drug concentrations on, e.g., chronic use, and are considered medically insignificant for the particular drug product studied.

For example, two antigen-binding proteins (or antibodies) are bioequivalent if there are no clinically meaningful differences in their safety, purity, or potency.

As another example, two antigen-binding proteins (or antibodies) are bioequivalent if a patient can be switched one or more times between the reference product and the biological product without an expected increase in the risk of adverse effects, including a clinically significant change in immunogenicity, or diminished effectiveness, as compared to continued therapy without such switching.

Two antigen-binding proteins (or antibodies) are also bioequivalent if they both act by a common mechanism or mechanisms of action for the condition or conditions of use, to the extent that such mechanisms are known.

Bioequivalence can be demonstrated by in vivo and/or in vitro methods. Bioequivalence measures include, e.g., (a) an in vivo test in humans or other mammals, in which the concentration of the antigen-binding protein or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antigen-binding protein (or its target) is measured as a function of time; and (d) a well-controlled clinical trial that establishes safety, efficacy, or bioavailability or bioequivalence of an antigen-binding protein.

Bioequivalent variants of the antigen-binding proteins (or antibodies) of the invention can be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins can include antigen-binding protein variants comprising amino acid changes, which modify the glycosylation characteristics of the antigen-binding proteins, e.g., mutations that eliminate or remove glycosylation.

Another aspect of the invention is directed to anticoagulant compositions comprising any of the antigen-binding proteins or bioconjugates disclosed herein and a physiologically acceptable carrier. By way of example and not of limitation, the anticoagulant composition can include antigen-binding proteins that bind to kringle-1 region of prothrombin in the open form and decrease conversion of prothrombin to thrombin, such as a humanized monoclonal antibody, a chimeric monoclonal antibody, a murine antibody, an Fab fragment, an F(ab′)2 fragment, a scFv fragment, a dAb, a VHH, or a conjugate of any of the above, or a combination of any of the above.

Any of the physiologically acceptable carriers can be used to formulate the compositions of the invention. Physiologically acceptable carriers have been amply described in a variety of publications, including, for example, “Remington: The Science and Practice of Pharmacy”, 19th Ed. (1995), or latest edition Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7.th ed., Lippincott, Williams, & Wilkins; and Handbook of Pharmaceutical Excipients (2000) A. H. Kibbe et al., eds., 3rd ed. Amer. Pharmaceutical Assoc.

The compositions can contain pharmaceutically acceptable auxiliary substances to approximate physiological conditions such as pH adjusting and buffering agents, toxicity adjusting agents and the like, for example, amino acids such as arginine and/or histidine, sodium acetate, sodium chloride, potassium chloride, calcium chloride, sodium lactate, hydrochloride, sulfate salts, solvates (e.g., mixed ionic salts, water, organics), hydrates (e.g., water), and the like.

While not being bound to a particular theory, the anticoagulant compositions can be used to treat or prevent a number of medical conditions or disorders associated with thrombi formation due to the ability of the antigen-binding proteins of the invention to decrease generation of prothrombin to thrombin. Accordingly, the anti-coagulant compositions of the invention can be used to treat or prevent deep vein thrombosis (DVT), pulmonary embolism (PE), atrial fibrillation (AFib), myocardial infarction (MI), ischemic stroke, unstable angina, hip or knee replacement surgery, COVID, antiphospholipid syndrome (APS), systemic lupus erythematosus (SLE), or cancer-associated thrombosis (CAT), in a patient in need thereof. The treatment can be prophylactic, i.e. administered to prevent the occurrence of thrombi generation, or to treat a patient already experiencing thrombi. Preferably, the patient is a human subject.

The dose of the antigen-binding protein in the anticoagulant composition, e.g., monoclonal antibody, or antigen-biding fragments thereof, can vary depending upon the age and the size of a subject, target disease, additional conditions, route of administration, and the like. When an antigen-binding protein of the invention is used for treating a disease or disorder in an adult patient, or for preventing such a disease, it is advantageous to administer the antigen-binding protein at a single dose of about 0.1 to about 60 mg/kg body weight, more preferably about 5 to about 60, about 20 to about 50, about 10 to about 50, or about 1 to about 10 mg/kg body weight. Depending on the severity of the condition, the frequency and the duration of the treatment can be adjusted. For example, the antigen-binding protein of the invention can be administered as an initial dose of about 0.1 mg to about 800 mg, about 1 to about 500 mg, about 5 to about 300 mg, about 10 to about 200 mg, or about 100 mg, or about 50 mg. The initial dose can be followed by administration of a second or a plurality of subsequent doses of the antigen-binding protein in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The injectable preparations can include dosage forms for intravenous, subcutaneous, intracutaneous, intracranial, intraperitoneal and intramuscular injections, drip infusions, etc. These injectable preparations can be prepared by methods publicly known. For example, the injectable preparations can be prepared, e.g., by dissolving, suspending or emulsifying the antigen-binding protein described above in a sterile aqueous medium or an oily medium conventionally used for injections. As the aqueous medium for injections, there are, for example, physiological saline, an isotonic solution containing glucose and other auxiliary agents, etc., which can be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which can be used in combination with a solubilizing agent such as benzyl benzoate, benzyl alcohol, etc. The injection is typically filled in an appropriate ampoule.

A pharmaceutical composition of the invention can be delivered intravenously or subcutaneously with a standard needle and syringe. In a number of embodiments, the administration is intravenous. In addition, with respect to subcutaneous delivery, a pen delivery device can be used. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and Ill (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the invention include, but certainly are not limited to the SOLOSTART pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.) and the HUMIRA™ Pen (Abbott Labs, Abbott Park, Ill.), to name only a few.

This invention is further illustrated by the following examples which should not be construed as limiting. The entire contents of all references, patents and published patent applications cited throughout this application are hereby incorporated herein by reference.

### EXAMPLES

The following non-limiting examples are provided to further illustrate the invention.

### Example 1

Prothrombin wild-type, prothrombin variants, and prothrombin fragments were produced as described previously (Chinnaraj et al., Front Med (Lausanne). 2018; 5:281, Chinnaraj et al., Blood Adv. 2019; 3(11):1738-1749). Immunization, ELISA, SPR, smFRET, and cryo-EM experiments are detailed at the end of the Examples section.

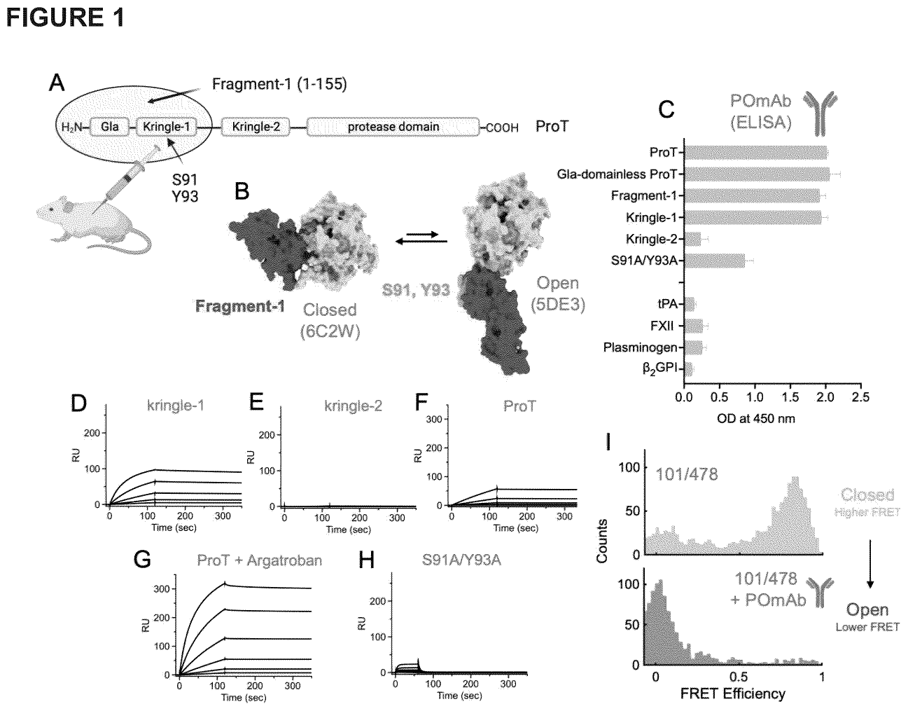

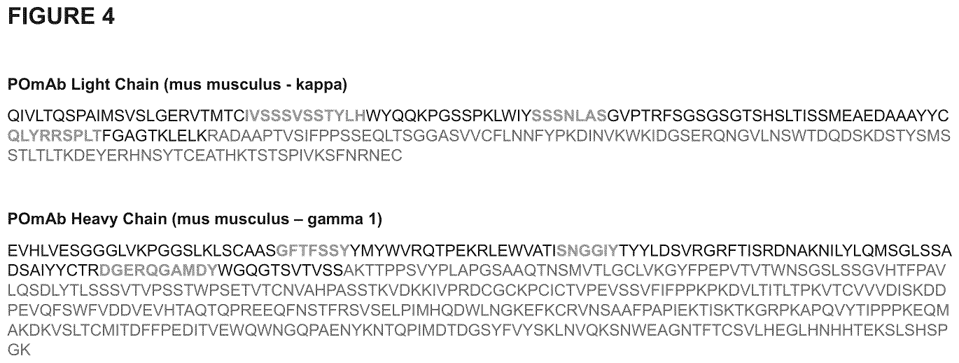

A library of new anti-prothrombin antibodies was generated by immunizing mice with fragment-1 of human prothrombin (FIG. 1, panel A). Antibodies were tested by ELISA against fragment-1, prothrombin, gla-domainless prothrombin, kringle-1, kringle-2, and the prothrombin variant S91A/Y93A, which carries two mutations in kringle-1, S91A and Y93A. Based on the previous analyses (Chinnaraj et al., et al., Front Med (Lausanne). 2018; 5:281) residues S91 and Y93 constituted an ideal epitope for a Type-I anti-PT antibody (with type I binding to the open form and type II binding to the closed form) as they were hidden in the closed form but exposed in the open structure of prothrombin (FIG. 1, panel B). One antibody, which was named POmAb (Prothrombin Open monoclonal Antibody, FIG. 4), reacted well against Fragment 1, gla-domainless prothrombin, and kringle-1 but showed a significantly reduced binding activity for the variant S91A/Y93A (FIG. 1, panel C). It also failed to interact with kringle-2 and other plasma proteins containing kringle domains, namely plasminogen, tissue plasminogen activator, and factor XII, as well as B2-glycoprotein I, a major antigen of antiphospholipid antibodies in APS (FIG. 1C). Together, these data indicate that POmAb is specific for kringle-1 of prothrombin.

After recombinantly expressing POmAb (FIG. 4), surface plasmon resonance (SPR) was used to confirm previous results obtained by ELISA and to determine affinity towards kringle-1. POmAb was found to be bound to kringle-1 with remarkably high affinity (Kd=3.7±1.5 nM) (FIG. 1, panel D, Table 1) and, as expected, it failed to bind structurally related kringle-2 (FIG. 1, panel E). POmAb was next tested for preference in binding to closed or open forms of prothrombin. To this end, SPR experiments were repeated using prothrombin in the absence (FIG. 1, panel F) and the presence of the active site ligand argatroban (FIG. 1, panel G). Argatroban was previously shown to shift the conformation of closed prothrombin to the open form, resulting in kringle-1 exposure. SPR documented that POmAb binds prothrombin with the same affinity as kringle-1 (Table 1).

TABLE 1
Binding kinetics of POmAb to prothrombin measured by SPR

| Species | kₒₙ (M⁻¹ s⁻¹) | K_{off} (s⁻¹) | K_{d} (nM) | ΔG (kcal/mol) |
|---|---|---|---|---|
| Kringle-1 | 6.91E+4 | 2.7E−4 | 3.70E−09 | −11.5 |
| Kringle-2 | n.d. | n.d. | n.d. | n.d. |
| ProT | 5.2E+4 | 1.3E−4 | 2.50E−09 | −11.7 |
| ProT + Argatroban | 4.03E+4 | 1.2E−4 | 2.98E−09 | −11.6 |
| ProTS91A/Y93A | 2.42E+4 | 2.9E−1 | 1.20E−05 | −6.7 |

Measurements were carried out in 20 mM Tris pH 7.4, 145 mM NaCl, 5 mM CaCl2, and 0.01% Tween20 at 25 C. Data were fit with a 1:1 binding model, which assumes there is no cooperativity between the two Fabs. Values reported are the average of three determinations. The standard deviation is no greater than 15%. The values of free energy AG were calculated using the formula ΔG=−RTInK, where R=1.9872 cal/K mol, T=298.15, and K is the equilibrium constant, which is 1/kd.

A second set of experiments was performed in the same manner and the results are shown below.

TABLE 2
Binding kinetics of POmAb to prothrombin measured by SPR

| | kₒₙ | K_{off} | K_{d} | Rmax | ΔG |
|---|---|---|---|---|---|
| Species | (M⁻¹ s⁻¹) | (s⁻¹) | (M) | (RU) | (kcal/mol) |
| Kringle-1 | 5.89e+04 | 3.14e−04 | 5.33e−09 | 117.4 | −11.3 |
| Kringle-2 | n.d. | n.d. | n.d. | n.d. | n.d. |
| ProT | 6.35e+03 | 1.22e−04 | 1.92e−08 | 106.1 | −10.5 |
| ProT + Argatroban | 3.66e+04 | 9.49e−05 | 2.59e−09 | 309.9 | −11.7 |
| ProTS91A/Y93A | 6.96e+04 | 7.02e−02 | 1.01e−06 | 32.1 | −8.2 |

Measurements were carried out in 20 mM HEPES PH 7.4, 145 mM NaCl, 5 mM CaCl₂), and 0.01% P20 at 25° C. The values of free energy AG were calculated using the formula ΔG=−RTInK, where R=1.9872 cal/K mol, T=298.15, and K is the equilibrium constant, which is 1/Kd.

However, POmAb binds significantly more prothrombin in the presence of argatroban because, under these circumstances, prothrombin is open. This indicates that the epitope recognized by POmAb is available in the open form of prothrombin but inaccessible in the closed form. SPR also confirmed that the binding of POmAb to the variant S91A/Y93A was reduced (FIG. 1, panel H) and established that the reason for the loss of binding is a fast dissociation rate, which is approximately three orders of magnitude faster compared to that of wild-type prothrombin. This implies that, without residues S91 and Y93, the antibody cannot remain bound to prothrombin.

To confirm the finding that POmAb binds to the open form, single-molecule FRET experiments were performed using the FRET pair 101-478 (FIG. 1, panel I). In the unbound form, 101-478 shows high FRET, which is a signature of the closed form. However, upon the addition of POmAb, the FRET signal shifted towards lower FRET. This indicates that POmAb interacts with kringle-1 of prothrombin, shifting the equilibrium towards the open form.

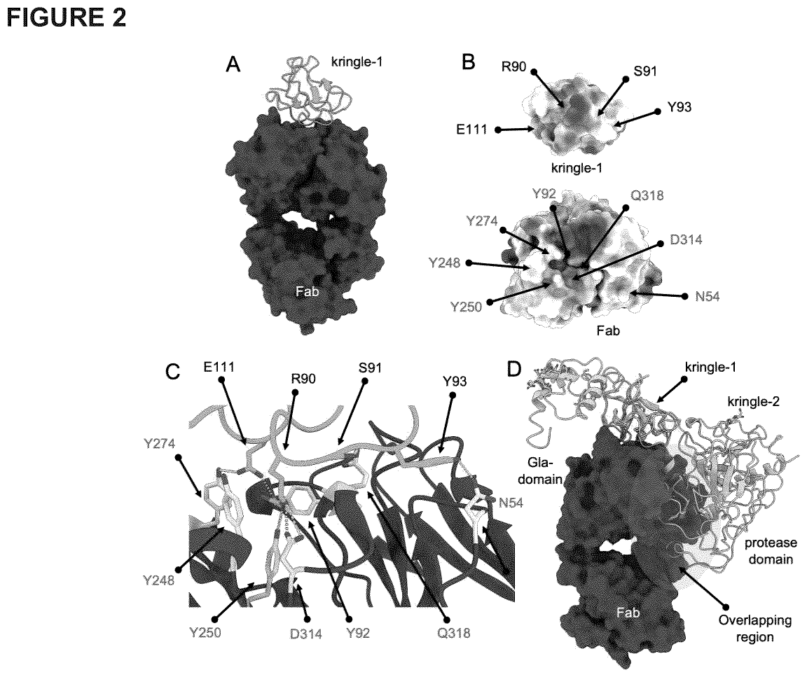

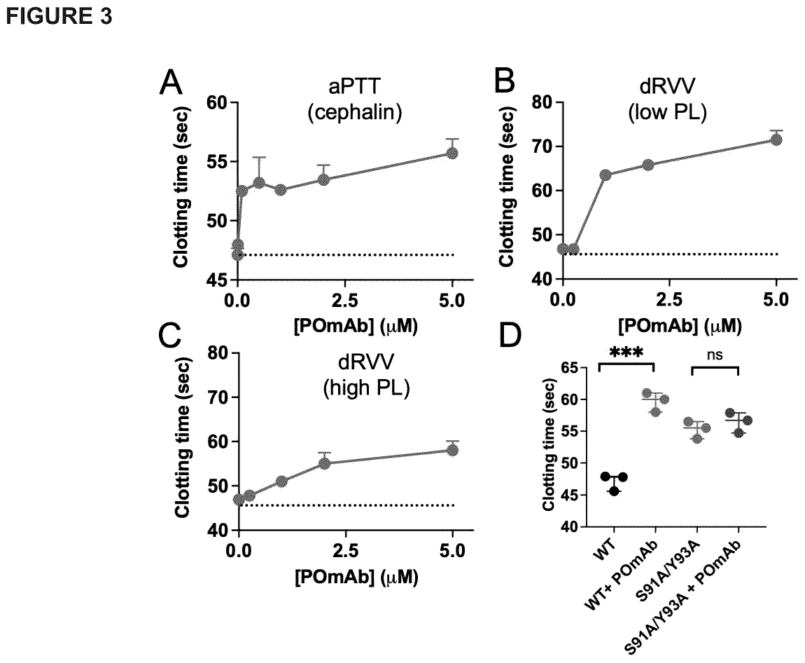

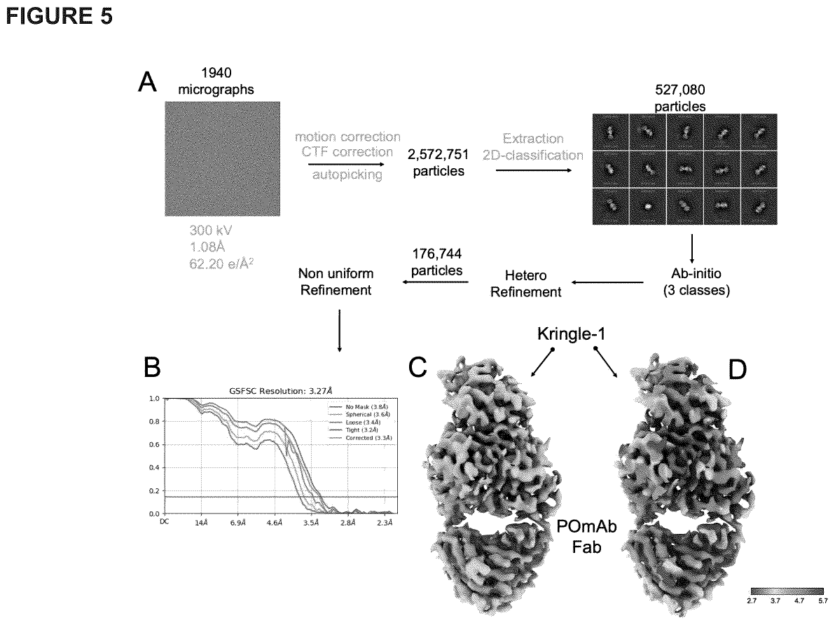

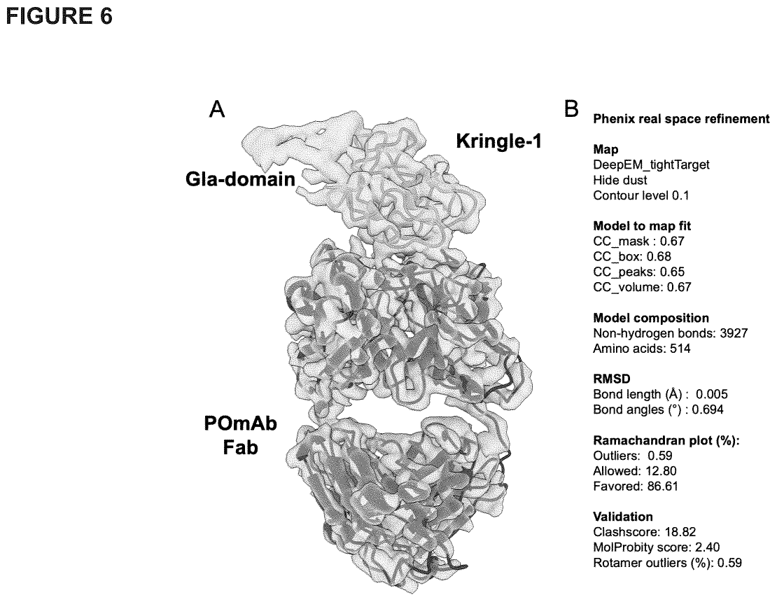

To visualize the binding interface, the cryo-EM structure of the Fab fragment of POmAb bound to prothrombin was solved (FIG. 2). Two-dimensional classification showed particles containing the Fab region of POmAb and a small portion of prothrombin, not the entire molecule (FIG. 5). This indicates that upon binding, prothrombin becomes flexible, except for the region bound by or adjacent to the Fab. In keeping with this interpretation, the density map displayed unequivocal densities for the POmAb Fab constant (heavy (HC) and light (HL)) and variable (heavy (VH) and light (VL)) domains and for kringle-1, with some weak density for the Gla-domain (FIG. 6). In contrast, no density was found for kringle-2 and the protease domain. The structure was solved at a nominal resolution of 3.2 Å (Table 3), with resolution at the binding interface approaching 2.7 Å (FIG. 5).

TABLE 3
Structure and model building refinement statistics

| Structure | POmAb/Kringle-1 |
|---|---|
| Data Accession | |
| PDB | |
| EMDB | |
| Data Collection | |
| Microscope | Titan Krios |
| Voltage (kV) | 300 |
| Automation Software | EPU |
| Detector | FEI Falcon IV (4k × 4k) |
| Nominal Magnification | 59000 |
| Pixel Size (Å/pixel) | 1.081 |
| Electron exposure (e⁻/Å²) | 62.2 |
| Spherical aberration (nm) | 0.01 |
| Defocus range (μm) | −1 to −2.4 μm |
| Micrographs collected | 1940 |
| Reconstruction | |
| Software | CryoSPARC (v4.21) |
| Micrographs Used | 1940 |
| Particle used in refinement | 176,744 |
| Symmetry | C1 |
| Overall resolution (Å) | 3.2 |
| FSC = 0.143 (masked) | |
| Local resolution range | 2.7~5.7 |
| Model Refinement | |
| Software | Phenix (v1.20.1) |
| Non-hydrogen bonds | 3927 |
| Protein Residues | 514 |

Analysis of the de-novo model built in the density revealed that the interface is centered around residues R90-Y93 of kringle-1 (FIG. 2, panel A) and stabilized by electrostatic interactions (FIG. 2, panel B), with a buried surface area of ˜850 Å2. Most complementarity determining regions (CDRs) participate in forming the binding interface. Key interactions occur between residues E111 in kringle-1 with Y92 and Y274 of the antibody and R90 with D314 in the CDR3 loop of the POmAb VH domain (FIG. 2, panel C). Residue Y93 of kringle-1 engages in hydrogen bonding with residue N54 in the CDR2 loop of the POmAb VL domain while S91 interacts with Q318 in the CDR3 loop of the POmAb VH domain, explaining why substitution of these residues to alanine result in loss of binding. The complete list of the intermolecular contacts is in Table 4.

TABLE 4
Contacts (<4.5Å) between POmAb and Kringle-1

| Chain C | Length | Chain B | Hydrogen | Salt |
|---|---|---|---|---|
| kringle-1 | (Å) | VH Ab | bond | bridge |
| Arg 90 [NE] | 2.6 | Asp 314 [OD2] | | X |
| Arg 90 [NE] | 3.7 | Asp 314 [OD1] | | X |
| Arg 90 [NE] | 3.3 | Asp 314 [OD2] | | X |
| Arg 90 [NH2] | 2.2 | Tyr 250 [NH2] | X | |
| Arg 90 [O] | 2.3 | Arg 317 [NH2] | X | |
| Ser 91 [OG] | 4.2 | Gln 318 [NE2] | X | |
| Tyr 93 [O] | 3.4 | Gln 318 [NE2] | X | |
| Gln 110 [OE1] | 2.7 | Tyr 248 [OH] | X | |
| Gln 110 [OE1] | 3.3 | Ser 267 [OG] | X | |
| Glu 111 [OE2] | 2.9 | Tyr 274 [OH] | X | |
| Chain C | Length | Chain A | Hydrogen | Salt |
| kringle-1 | (Å) | VL Ab | bond | bridge |
| Leu 88 [N] | 3.4 | Tyr 33 [OH] | X | |
| Tyr 93 [OH] | 2.3 | Asn 54 [OD1] | X | |
| Arg 90 [NH2] | 3.1 | Tyr 92 [OH] | X | |
| Cys 86 [O] | 2.2 | Tyr 33 [OH] | X | |
| Glu 111 [OE1] | 2.3 | Tyr 92 [OH] | X | |

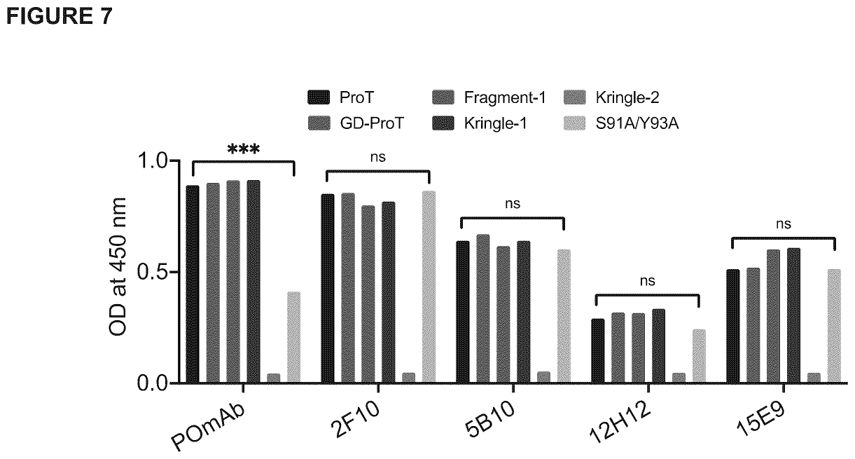

Superposition of the cryo-EM structure of POmAb bound to kringle-1 with the x-ray crystal structure of the closed form of prothrombin previously solved reveals why POmAb cannot bind with this conformation (FIG. 2, panel D). By interacting with kringle-1, the antibody clashes against the protease domain, forcing prothrombin to open. The cryo-EM structure suggests that targeting kringle-1 away from the region R90-Y93 might preserve or even stabilize the closed form. At the same time, Type I antibodies could engage residues other than those in the region R90-Y93, resulting in open conformations. The immunization experiments revealed several clones that, like POmAb, specifically interact with kringle-1 but, unlike POmAb, are insensitive to the mutations S91A and Y93A (FIG. 7).

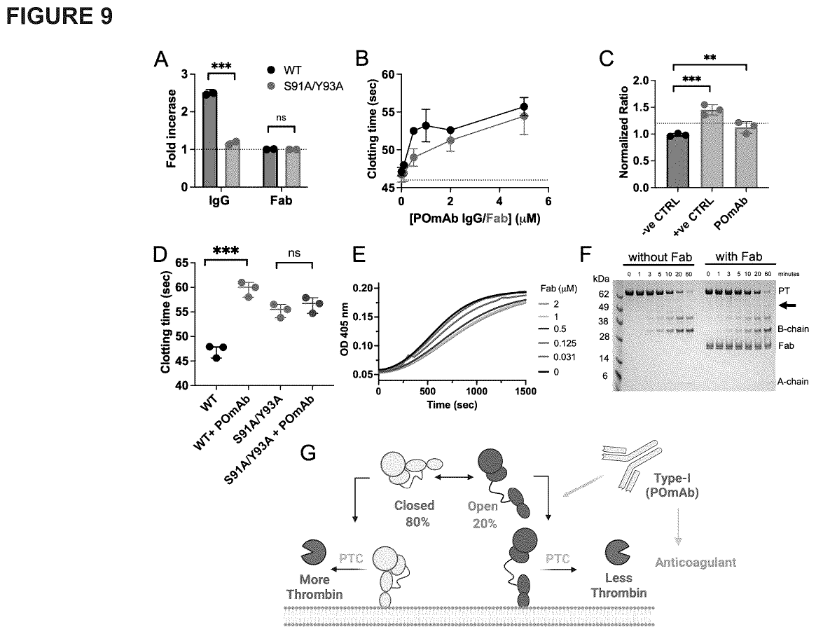

Some antibodies against prothrombin (i.e., aPS/PT) are known to have a strong correlation with lupus anticoagulant (LA), a laboratory test that indicates the phospholipid-dependent prolongation of the clotting time, which can be rescued by additional phospholipids. LA is believed to occur due to antibody-mediated dimerization of prothrombin, which competes with the binding of clotting factors for phospholipid surfaces (Simmelink et al. Br J Haematol. 2001; 113(3):621-629; Noordermeer et al. J Thromb Haemost. 2021; 19(4):1018-1028; Field S L et al. J Immunol. 2001; 166(10):6118-6125). As shown in FIG. 9, panel A, it was found that lgG dramatically increased the binding of prothrombin wild-type to negatively charged liposomes but not the variant S91A/Y93A, which is weakly bound by POmAb. This effect was not seen with the Fab fragment, which, unlike IgG, is monovalent. However, both IgG and Fab resulted in a significant dose-dependent prolongation of the clotting time initiated by the activators cephalin (FIG. 9, panel A) and diluted Russel viper venom (FIG. 9, panel B). However, this effect was only partly corrected by the addition of excess phospholipids (FIG. 9, panel C). This indicates that POmAb works as an anticoagulant in human plasma but produces a modest LA phenomenon. To verify that this prolongation effect was due to specific binding to prothrombin, prothrombin-deficient plasma supplemented with either prothrombin wild-type or the variant S91A/Y93A was tested (FIG. 9, panel D). As expected, this prolongation effect was only present with prothrombin wild type but not with the variant S91A/Y93A.

The discovery that Fab of POmAb has an anticoagulant effect similar to that of the whole IgG was significant. It shows that the mechanism resulting in the prolongation of clotting time is more complex than just sequestration of phospholipids. To investigate this further, prothrombin conversion to thrombin was monitored in vitro using purified components. In a continuous kinetic assay as described by Chinnaraj et al., Sci Rep., 8(1):2945, it was found that Fab POmAb inhibited prothrombin conversion to thrombin by prothrombinase in a dose-dependent manner (FIG. 9, panel E). However, inhibition never reached completion, even when excess Fab POmAb was used compared to prothrombin. Prothrombin was cleaved by fXa at residues R155, R271, and R320, to generate thrombin as previously shown in the art. Gel-electrophoresis experiments in FIG. 9, panel F confirmed the inhibitory effect of Fab POmAb and showed an altered activation pathway, with more cleavage at R155. Thus, Fab of POmAb lowered thrombin generation by modulating prothrombin conformation rather than blocking its interaction with prothrombinase (FIG. 9, panel G).

In addition to proving that POmAb exerts an anticoagulant effect in human plasma, data in FIG. 9, panel G provides insights into the mechanism of action and advances a new concept about how to reduce thrombin generation. Plasma supplemented with prothrombin wild-type bound to POmAb, which is in the open form, takes almost the same time to clot as plasma supplemented with S91A/Y93A, which is also open. However, when compared to plasma supplemented with prothrombin wild type alone, which is mostly closed, it takes longer to clot. It has been previously shown that the open form is converted to thrombin more slowly because cleavage at R320 by prothrombinase is reduced in favor of cleavage at R271 (Chinnaraj et al., Front Med (Lausanne). 2018; 5:281, Chinnaraj et al. Sci Rep. 2018; 8(1):2945). Additionally, Ruben et al. documented that the interaction of prothrombin with prothrombinase complex requires minimal contact between the protease domain of prothrombin and prothrombinase (Ruben et al., Blood. 2022; 139(24):3463-3473). These observations indicate that POmAb prolongs the clotting time by altering the open/closed equilibrium of prothrombin rather than preventing binding of prothrombin to prothrombinase complex. Further development of POmAb and other Type I anti-PT antibodies can lead to a new class of anticoagulants with a unique mechanism of action.

Recent studies have shown that murine monoclonal anti-PT antibodies can activate human platelets via FcγRIIA (Chayoua et al., J Thromb Haemost. 2021; 19(7):1776-1782) only if they exert LA in human plasma. In fact, anti-PT antibodies devoid of LA could not activate platelets in the same study. These findings imply that POmAb exerts a prothrombotic effect using different mechanisms compared to previously published platelet-stimulating antibodies. While not being bound to a theory, POmAb appears to be protective as it lowers thrombin generation without activating platelets.

Chemicals. Analytical-grade chemicals were purchased from Sigma-Aldrich, ThermoFisher, and Cytiva. Buffers were sterile filtered at 0.2 μm before use. Nu-PAGE 4-12% Bis-Tris gels, SeeBlue plus2 pre-stained protein standard, and Coomassie blue solution were from ThermoFisher.

Production and purification of recombinant proteins and prothrombin fragments. Residues of the FII gene (Uniprot P00734) corresponding to residues 1-579 of the mature protein, were cloned in a pDEST40 expression vector using the Gateway cloning technology (Life Technologies). A tag (YLEDQVDPRLIDGK) was added at the C-terminus to facilitate purification. The mutations S91A and Y93A were generated using the Quickchange Lighting kit (Agilent) and appropriate primers (Integrated DNA Technologies). After sequencing, the recombinant proteins were expressed in Expi293 cells (Thermo Fisher Scientific) in the presence of vitamin K and purified by affinity chromatography, as detailed in Chinnaraj et al. Sci Rep. 2018; 8(1):2945, Chinnaraj et al., Front Immunol. 2021; 12:741589.

The cDNA of light and heavy chains of POmAb were cloned into pcDNA 3.4 as a mouse IgG1 (FIG. 4). The antibody was recombinantly produced in CHO cells as per manufacturing instructions. Purification was performed using protein A, followed by size exclusion chromatography on a Superdex 200 Increase 10/300 GL (Cytiva) equilibrated in 20 mM Tris-HCl pH 7.4, 145 mM NaCl. The purity of each protein was verified by Coomassie-stained 4-12% SDS-PAGE (NuPAGE, Invitrogen). Antibody 5B10 was produced directly from hybridoma cells obtained from immunization experiments. Antibody AHP, a type II antibody, is commercially available from Prolytix (AHP-5013). The purity of each protein was verified by Coomassie-stained 4-12% SDS-PAGE (NuPAGE, Invitrogen). Proteins were aliquoted and stored at −80° C. until used.

Prothrombin purified from human plasma (1-579), Gla-domainless prothrombin (amino acids 44-579), and fragment-1 (amino acids 1-155) purified were obtained from Enzyme Research Laboratories (IN, USA). Kringle-1 (amino acids 44-155) and kringle-2 (amino acids 156-271) were obtained by limited proteolysis of GD-prothrombin using a combination of thrombin and factor Xa, as described earlier (Chinnaraj et al., Blood Adv. 2019; 3(11):1738-1749). Protein concentrations were determined by reading at 280 nm with molar extinction coefficients adjusted based on the amino acid sequence. All other chemicals were purchased from Sigma-Aldrich.

Immunization experiments. BALB/c mouse immunization experiments were performed by Genescript Inc. using the MonoBoost protocol. Human fragment-1 (aa 1-155) was prepared in 20 mM Tris-HCl PH 7.4, 145 mM NaCl, and 5 mM CaCl₂). Screening of clones resulting from cell fusion against immobilized human prothrombin, prothrombin fragments, and prothrombin variants was performed using an in-house enzyme-linked immunoassay (ELISA) in which each protein of interest was immobilized into Nunc MAXISORP (Thermo Fisher Scientific) plates at 0.5 μg/well and binding was detected using 1:10,000 dilution of HRP-conjugated anti-mouse IgG (γ-chain specific) antibody (Sigma Aldrich).

Enzyme-linked immunoassay of kringle-containing proteins and B2GPI. One hundred microliters of 5 μg/ml prothrombin, plasminogen, tissue plasminogen activator, FXII, and β2GPI (Enzyme Research Laboratories) solubilized in 0.1 M sodium bicarbonate pH 9.6 were added to a Nunc MAXISORP plate and incubated overnight at 4° C. After washing three times with 200 μl/well of 20 mM Tris-HCl, PH 7.4, 145 mM NaCl, 5 mM CaCl2, Tween 20 0.05% (TBSC-T), wells were blocked with 200 μl of 20 mM Tris-HCl, pH 7.4, 145 mM NaCl, 1% BSA (TBS-B) for 60 min at room temperature. One hundred microliters of POmAb (50 μg/ml), which is >1000-fold higher than the EC50 value of POmAb for prothrombin, prepared in the sample buffer 20 mM Tris-HCl, pH 7.4, 145 mM NaCl, 5 mM CaCl2, Tween 20 0.05%, 1% BSA (TBSC-BT) were added to each well and incubated for 60 min at room temperature. Plates were washed three times TBSC-T, and then 100 μl of 1:10,000 dilution of HRP-conjugated anti-mouse IgG (γ-chain specific) antibody (Sigma Aldrich) was added for 60 min at room temperature. Plates were washed three times with TBS-T and then incubated with 100 μl of 3,30,5,50-tetramethylbenzidine (TMB) liquid substrate (Sigma Aldrich). After 30 min, the colorimetric reaction was quenched with 100 μl of TMB-stop solution. The optical density at 450 nm was recorded using a SPARK microplate reader (TECAN). Data were plotted and analyzed using ANOVA in Prism 9.0.

TABLE 5
EC₅₀ values of POmAb, 5B10, and AHP for
prothrombin wild-type, prothrombin mutants,
and prothrombin fragments measured by ELISA

| | POmAb | 5B10 | AHP |
|---|---|---|---|
| Species | (μg/ml) | (μg/ml) | (μg/ml) |
| Kringle-1 | 0.020 ± 0.005 | 0.038 ± 0.006 | Unstable |
| Kringle-2 | Unstable | Unstable | 0.249 ± 0.05 |
| ProT | 0.013 ± 0.003 | 0.031 ± 0.004 | 0.195 ± 0.07 |
| ProTS91A/Y93A | 0.301 ± 0.07 | 0.038 ± 0.007 | 0.185 ± 0.08 |

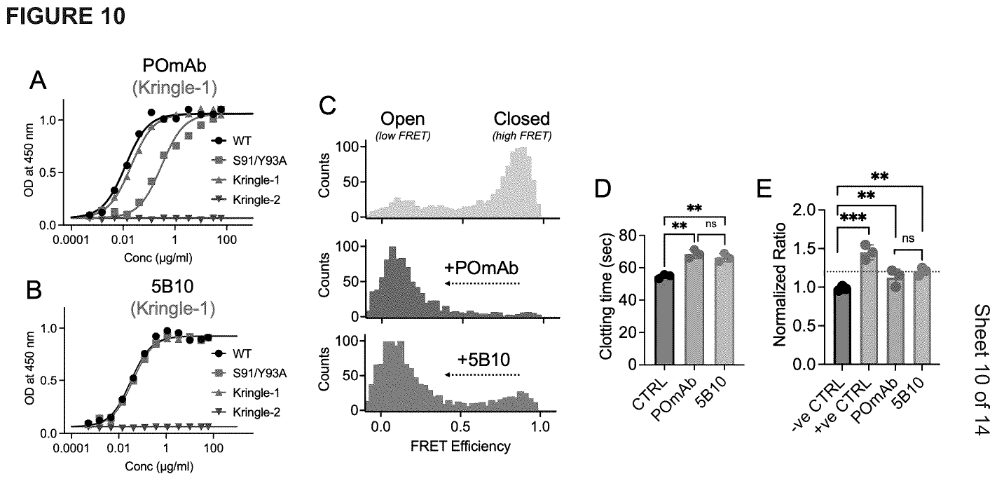

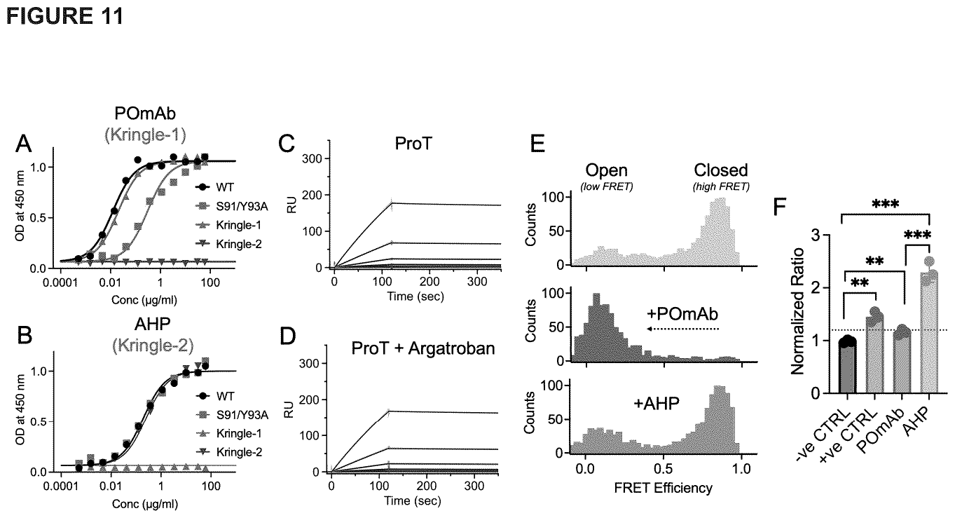

EC₅₀ values were determined by fitting dose-dependent experiments in FIG. 10 and FIG. 11 with a Hill equation. They are reported in μg/ml+/−standard deviation calculated from the residuals of the fit.

TABLE 6
Binding kinetics of AHP to prothrombin measured by SPR

| | kₒₙ | K_{off} | K_{d} | Rmax | ΔG |
|---|---|---|---|---|---|
| Species | (M⁻¹ s⁻¹) | (s⁻¹) | (M) | (RU) | (kcal/mol) |
| Kringle-1 | n.d. | n.d. | n.d. | n.d. | n.d. |
| Kringle-2 | 4.60e+03 | 1.28e−04 | 2.78e−08 | 399.7 | −10.3 |
| ProT | 3.54e+03 | 1.36e−04 | 3.83e−08 | 516.8 | −10.1 |
| ProT + Argatroban | 3.27e+03 | 1.22e−04 | 3.74e−08 | 522.3 | −10.1 |
| ProTS91A/Y93A | 6.32e+03 | 1.23e−04 | 1.95e−08 | 424.5 | −10.5 |

Measurements were carried out in 20 mM HEPES pH 7.4, 145 mM NaCl, 5 mM CaCl₂), and 0.01% P20 at 25° C. The values of free energy AG were calculated using the formula ΔG=−RTInK, where R=1.9872 cal/K mol, T=298.15, and K is the equilibrium constant, which is 1/Kd.

Surface Plasmon Resonance (SPR) experiments. Binding affinities for POmAb were measured using CM5 sensor chip in which POmAb was immobilized at 3000 RU using NHS/EDC chemistry. Titrations were performed by injecting increasing concentrations of prothrombin, prothrombin fragments, and prothrombin variants in running buffer (20 mM Tris pH 7.4, 145 mM NaCl, 0.01% w/w Tween20) at a flow rate of 25 μl/min at 25° C. All experiments were carried out using a BIAcore-S200 instrument (GE-Healthcare). After correction for baseline, association (kₒₙ) and dissociation (k_{off}) rate constants were obtained using a 1:1 model provided by BIAevaluation software. Data reported in the examples represents the average of three independent experiments.

Phospholipid-binding experiments were performed on a BIAcore-S200 instrument (GE-Healthcare) at 25° C., as described previously (Chinnaraj et al., Blood Adv. 2019; 3(11):1738-1749, Kumar et al. J Biol Chem. 2021; 297(2):100890). Liposomes composed of 100% POPC and 80:20% (molar ratio) POPC:POPS prepared by extrusion using 100 nm polycarbonate membranes (Avanti) were immobilized onto different microfluidic channels of an L1 chip (final RU was 1250 for POPC and 850 for POPC:POPS) and running buffer composed of 20 mM Tris pH 7.4, 150 mM NaCl, 5 mM CaCl2, 0.1% w/w BSA run overnight to stabilize baseline. After performing preliminary titration experiments with prothrombin wild-type and variant S91A/Y93A to confirm the functional integrity of the immobilized liposomes and analytes, prothrombin wild-type (250 nM) and the variant S91A/Y93A (250 nM) were mixed with increasing concentrations of IgG POmAb or Fab (0-10 g/ml) and the solutions injected over liposomes coated microfluidic channels at 25 μl/min to measure binding. Regeneration buffer between runs was 20 mM Tris-HCl PH 7.4, 1.5 NaCl, and 2.5 mM EDTA.

Single-molecule FRET. smFRET measurements of freely diffusing prothrombin molecules labeled at residues 101 and 478 with the FRET pair Alexa555/Alexa647 were performed with a confocal microscope MicroTime 200 (PicoQuant, Berlin, Germany) equipped with pulsed interleaved excitation, as reported previously (Chinnaraj et al. Sci Rep. 2018; 8(1):2945, Chinnaraj et al., Front Immunol. 2021; 12:741589, Pozzi et al. J Biol Chem. 2016; 291(35):18107-18116). Samples prepared at 80 pM in 20 mM Tris-HCl PH 7.4, 150 mM NaCl, a 5 mM CaCl₂), and 0.01% Tween 20 were collected for 30 minutes at room temperature in the absence and presence of 500 nM POmAb.

Production and functional validation of Fab fragment. Fab fragment of POmAb was produced using Pierce™ Fab Micro Preparation Kit and further purified on a Superdex 200 increase 10/300 GL column equilibrated in 20 mM Tris-HCl PH 7.4, 150 mM NaCl, and 5 mM CaCl2.

Cryo-EM grid preparation and data collection. Fab of POmAb in complex with prothrombin forced in open conformation by the addition of 300 μM argatroban was purified on size-exclusion chromatography using a Superdex 200 increase 10/300 GL column in the buffer of 20 mM Tris-HCl PH 7.4, 150 mM NaCl, 5 mM CaCl₂). Fractions containing the complex verified by SDS-PAGE were pooled and concentrated to 0.88 mg/ml by centrifugation using a 30 kDa cutoff.

At the time of freezing, grids were plasma cleaned on a Gatan Solarus 950 for 60 seconds. After testing different concentrations, final grids were prepared by applying 3 μl of 0.33 mg/ml the complex onto a 300-mesh Quantifoil 2/2holey grids (Quantifoil Electron Microscopy Sciences) and blotted for 2 seconds, and then immediately plunge frozen in liquid ethane on a FEI Vitrobot Mark IV. Grids were loaded onto a Titan Krios G3 cryo-TEM operating at 300 kV equipped with a FEI Falcon IV (4k×4k) direct electron detector. Screening and data collection were performed at a pixel size of 1.081 Å, using a dose of 62.2 e⁻/Å2 across 40 frames, using a set defocus range of −1 to −2.4 μm. A total of 1940 micrographs were collected using EPU.

Cryo-EM image processing and analysis. Micrographs were imported and processed using cryoSPARC v4.21 as described in Punjani et al., Nat Methods. 2017; 14(3):290-296 (FIG. 5). Micrographs were aligned and dose-weighted using patch motion correction, and the micrograph contrast transfer function (CTF) parameters were estimated using patch CTF estimation. Particles were automatically picked using a blob picker function, extracted into 256-pixel boxes, and subjected to an initial round of 2D classification to assess the quality of the particles and orientation biases. Several classes capturing different orientations of the complex were used to create an initial ab-initio 3D model, whose particles were subsequently used as templates for picking more higher-quality particles. Particles re-extracted using this strategy underwent multiple rounds of 2D classification and heterogeneous refinement followed by a non-uniform refinement with C1 symmetry. Local and global CTF refinements followed by a final round of non-uniform refinement provided a consensus overall resolution of 3.27 Å as per gold-standard Fourier shell correlation cutoff of 0.143. This is an excellent result considering that the overall complex accounts for 60 kDa and that cryo-EM studies of monomeric proteins with molecular weight lower than 100 kDa remain challenging (see, e.g, Wentinck et al., Curr Res Struct Biol. 2022; 4:332-337, Herzik et al., Nat Commun. 2019; 10(1):1032). Such a resolution (about 3 Å or better) is needed for reliable atomic interpretation. Map examination and visualization were conducted using USCF Chimera and Chimera X (v 1.6). Unsharpened, sharpened, and half maps have been deposited in EMDB with accession code EMD-42185.

Model building and refinement. The 3.27 Å map of the complex was post-processed using the DeepEMhancer script (see, e.g., Sanchez-Garcia et al., Commun Biol. 2021; 4(1):874) and the output file was used for atomic model building and refinement. The map generated using DeepEMhancer was deposited as the primary map in EMDB with accession code EMD-42185

The antigen (prothrombin) and antibody (Fab of POmAb) were initially docked and fit into the map as a rigid body using UCSF Chimera. The protein coordinates for the Fab of POmAb were obtained by modeling the antibody given its aminoacidic sequence using I-TASSER (see, e.g., Zhang Y., BMC Bioinformatics. 2008; 9:40). The protein coordinates for the kringle-1 of human prothrombin (residues 62-145) were extracted from the crystal structure of human prothrombin variant Δ146-167(4o03.pdb) (see, e.g., Pozzi et al., Proc Natl Acad Sci USA. 2014; 111(21):7630-7635), which was solved in the presence of CaCl2 at 3.38 Å resolution. This is the only structure of human prothrombin solved in the presence of CaCl₂). The initial model underwent several rounds of refinement using phenix.real_space_refine (Phenix v 1.20.1-4487) followed by manual adjustments using COOT (v 0.9.8.8). Secondary structure restraints for Fab were obtained using Fab structure 517X solved at 1.85 Å resolution by X-ray crystallography as described in Naschberger et al., Acta Crystallogr D Struct Biol. 2016; 72(Pt 12):1267-1280. Atomic models were validated using the comprehensive cryo-EM validation script in Phenix, which includes MolProbity and Model vs. Data correlation coefficients. Model visualizations were conducted using Chimera X and PyMol software (Schrödinger, LLC). The model refinement statistics of the cryo-EM structure are summarized in FIG. 6. The final model containing kringle-1 bound to Fab of POmAb has been deposited in PDB with accession code 8UF7.

Activated Partial Thromboplastin Time. aPTT was measured using the CEPHEN APTT (ACK512K-RUO) kit on an ST4 semiautomated coagulometer (Diagnostica Stago, Gennevilliers, France). Briefly, 50 μl of citrated plasma was mixed with 50 μl of antibody (0-5 μM) or vehicle (20 mM Tris-HCl PH 7.4, 150 mM NaCl) and 50 μl aPTT reagent solubilized in water in the appropriate cuvette. After 180 seconds at 37° C., the reaction was started by adding 50 μl of 25 mM CaCl₂). Similarly, 50 μl of citrated prothrombin-deficient plasma (Hematologic Technologies) was mixed with 50 μl prothrombin wild-type (0.5 μM) or variant S91A/Y93A (0.5 M), antibody (5 M) or vehicle and 50 μl aPTT reagent in the appropriate cuvette. After 180 seconds at 37° C., the reaction was started by adding 50 μl of 25 mM CaCl₂).

Diluted Russel Viper Venom. dRVV was measured using the HEMOCLOT LA-S (ACK090K-RUO) and LA-C (ACK091K-RUO) kits on an ST4 semiautomated coagulometer (Diagnostica Stago, Gennevilliers, France). Briefly, 50 μl of citrated plasma was mixed with 50 μl of antibody (μM) in the appropriate cuvette. After 180 seconds at 37° C., the reaction was started by adding 100 μl of dRVV reagent. Normal control plasma (A223201-RUO, −ve control) and LA-control plasma (ASC081K-CAN, +ve control) were purchased from Aniara (USA). Normalized ratio was calculated as recommended by the manufacturer.

Activation of Prothrombin. Prothrombin activation was monitored using a colorimetric assay that continuously reports the amount of thrombin that is generated upon cleavage by the prothrombinase complex as described by Pozzi N et al., J Biol Chem. 2016; 291(35):18107-18116 and Pozzi N et al., J Biol Chem. 2016; 291(35):18107-18116. Briefly, prothrombin (250 μl, 25 nM) bound to Fab POmAb (0-2 μM) was reacted with 2.5 pM factor Xa, 20 μM phospholipids, 2 nM cofactor Va, and 24 μM chromogenic substrate FPF-pNA. Data were collected on a SPARK microplate reader (TECAN). Data were plotted using Prism 9.0.

Prothrombin activation was also monitored by SDS-PAGE in the presence of 10 μM dansylarginine-N-(3-ethyl-1,5-pentanediyl)amine (DAPA), as detailed in Pozzi N et al., J Biol Chem. 2016; 291(35):18107-18116 and Pozzi N et al., J Biol Chem. 2016; 291(35):18107-18116. Briefly, prothrombin (0.09 mg/ml, 1.25 M) dissolved in 145 mM NaCl, 20 mM Tris pH 7.4, and 5 mM CaCl₂) was activated with the enzyme fXa (0.2 nM), phospholipids (POPC:POPS 80:20, 20 UM) and cofactor Va (10 nM) in the absence or presence of Fab POmAb (1.5 M). Following the addition of the enzyme, samples (25 μl, 2.2 μg) were quenched at different time intervals with 10 μl of NuPAGE LDS buffer with β-mercaptoethanol as the reducing agent and 20 mM EDTA. Samples were processed by NuPAGE Novex 4-12% Bis-Tris protein gels run with MES buffer. Protein bands were stained using SimplyBlue SafeStain. Images were taken with a CCD camera and shown without further processing.

Thrombin Generation Assays. Calibrated thrombin generation assays were performed according to established procedures (Pontara E, et, al. J Thromb Haemost. 2021; 19:805-13, and Pontara E, et al. J Thromb Haemost. 2023; 21:3138-44).

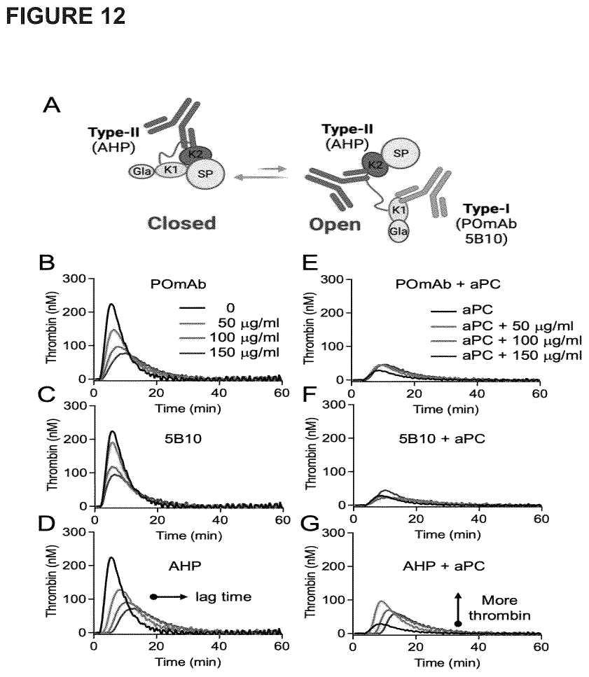

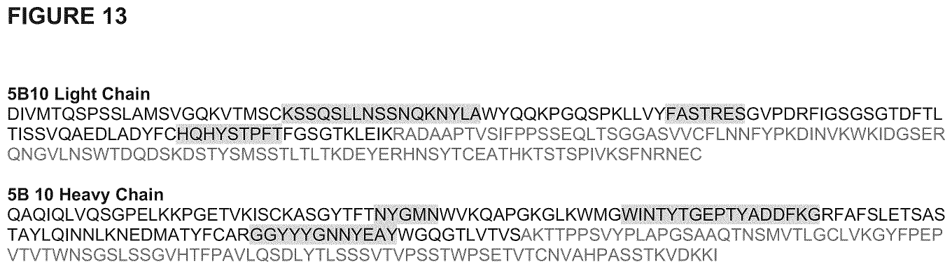

Briefly, 60 μl of normal pooled plasma was mixed with 20 μl PPP reagent (5 pM tissue factor/4 μM phospholipids, Diagnostica Stago) and 20 μl of buffer (0) or 50, 100, and 150 μg/ml of POmAb, 5B10, and AHP. These concentrations were chosen based on previous studies with anti-prothrombin antibodies purified from APS patients (Pontara E, et, al. J Thromb Haemost. 2021; 19:805-13). Solutions were incubated for 10 minutes at 37° C. before starting the reaction with 20 μl of a mixture containing a thrombin-specific fluorescent substrate and calcium chloride (FluCa-kit reagent, Diagnostica Stago). Thrombin generation was monitored in real-time, and the Thrombinoscope software was used to analyze the kinetic curves, employing a thrombin calibration curve to convert fluorescence signal into thrombin concentrations (nM). A commercially available human IgG1 that does not react with plasma proteins was utilized as a control (Syd Labs). When specified, 10 nM aPC (Diagnostica Stago) was added for 10 minutes before initiating the reaction, as done before. The results of these experiments are presented in FIG. 12 and Table 7.

Without aPC, all antibodies reduced thrombin generation, but they did so through different mechanisms. Specifically, POmAb (FIG. 12, panel B) and 5B10 (FIG. 12, panel C) reduced the thrombin peak without significantly prolonging the lag time. In contrast, AHP (FIG. 12, panel D), besides decreasing the thrombin peak, showed a significant prolongation of the lag time, indicating a more profound anticoagulant effect. This further supports the theory that AHP, but not POmAb and 5B10, exhibit a strong lupus anticoagulant effect as measured by Russell Viper Venom Time.

In the presence of aPC, major differences between the antibodies were observed. Without antibodies, adding aPC led to a significantly reduced thrombin generation by approximately 80%. Similar reductions were observed with POmAb (FIG. 12, panel E) and 5B10 (FIG. 12, panel F). However, for AHP, the thrombin peak was much higher, implying reduced anticoagulant function of aPC (FIG. 12, panel G). Thus, AHP, but less so POmAb or 5B10, causes significant aPC resistance under these experimental conditions. Moreover, there was a clear dose-dependent effect, indicating that AHP drives aPC resistance in a concentration-dependent manner. While not being bound to a theory, a possible mechanism for aPC resistance is if antibodies block the anticoagulant activity of aPC by binding to aPC, its cofactor protein S, its substrates factor V/Va and factor VIII/Villa or other abundant proteins, such as β2-glycoprotein I, which can modulate aPC activity. The data are shown in the Table 7 below.

TABLE 7
Thrombin generation parameters in the absence and presence of 10 nM activated
protein C (aPC) at the highest concentration of antibody tested.

| | Without aPC | | | | With aPC | | | |
|---|---|---|---|---|---|---|---|---|
| | CTRL | POmAb | 5B10 | AHP | CTRL | POmAb | 5B10 | AHP |
| Lag time (min) | 2.3 | 3.7 | 2.3 | 6.3 | 4.3 | 5.7 | 5.3 | 9.3 |
| ETP (nM · min) | 1603 | 1069 | 1071 | 1039 | 292 | 607 | 488 | 715 |
| Peak (nM) | 225 | 76.3 | 94.1 | 71.2 | 28.4 | 46.0 | 44.3 | 60.0 |
| Time to Peak (min) | 5.7 | 9.7 | 6.3 | 12.3 | 8.7 | 10.6 | 10.3 | 13.6 |
| VeI Index (nM/min) | 67.5 | 12.7 | 23.5 | 11.9 | 6.5 | 9.2 | 8.8 | 13.8 |

Parameters in the Table 7 are defined as follows: 1) lag time (min) is the time between the addition of the trigger and the initiation of the thrombin generation curve; 2) the endogenous thrombin potential (ETP; nM·min) represents the amount of thrombin formed over 60 minutes, 3) peak (nmol/L) is the highest thrombin concentration that can be generated, 4) time to peak (min) represents the time needed to reach the peak level; 5) the velocity index (VI, nM/min) is the slope between lag time and time to peak. Each data point was repeated twice, obtaining very similar results. The data in Table 7 are the average of these two determinations. Errors were less than 10%.

Testing of POmAb (0.5 mg/kg) in vivo. Male 8- to 10-week-old C57BL6/J mice (Strain #000664, Jackson Laboratory) were anesthetized with a ketamine and xylazine cocktail administered by intraperitoneal injection. An indwelling catheter was placed in the jugular vein to allow for further intravenous administration of ketamine to maintain the mouse in an appropriate plane of anesthesia and for subsequent administration of fluorescently labeled antibodies. A testis was externalized from the scrotum and secured to a custom intravital imaging tray using a 28G needle. Excess connective tissue and fat were removed from the testis using a pair of fine-tipped forceps. An incision was made in the cremaster muscle extending proximally from the base of the testis allowing the cremaster muscle to be pinned out over a cover slip mounted in the intravital imaging tray. The cremaster muscle was super-perfused with 37° C. bicarbonate buffered saline aerated with 95% N2 and 5% CO₂. Mice were administered with Dylight647 labeled anti-CD42b (0.1 mg/g body weight; Emfret) and Dylight488 labeled anti-fibrin (clone 59D8, 0.5 mg/g body weight) by intravenous infusion through the indwelling jugular catheter. Either POmAb labeled with Dylight405 or a non-reactive mAb control was added by intravenous infusion at 0.5 mg/kg 10 minutes prior to the first observation.

Cremaster arterioles were subject to injury using a 170 μJ pulsed nitrogen dye laser at 440 nm using the Micropoint laser system (Andor). The pulse was directed at a point just inside the lumen of the vessel adjacent to the vessel wall, creating an average injury size with a length of 56±28 μm (n=48 injuries in 4 mice) for control animals and 53±13 μm (n=40 injuries in 4 mice) for POmAb treated animals.

Accumulation of fluorescent signal at the site of thrombus was visualized through a 60×1.0 NA water immersion objective (Olympus) mounted to an AX-70 fluorescence microscope (Olympus) equipped with a CCD camera (ORCA Flash 4.0, Hamamatsu Photonics). Images were captured in four channels (Brightfield, 405/420, 488/520 and 640/670 nm) for 240 seconds at 2 frames/second using a digital camera. Data acquisition and analysis were conducted using SlideBook software (v6.0, Intelligent Imaging Innovations, CO).

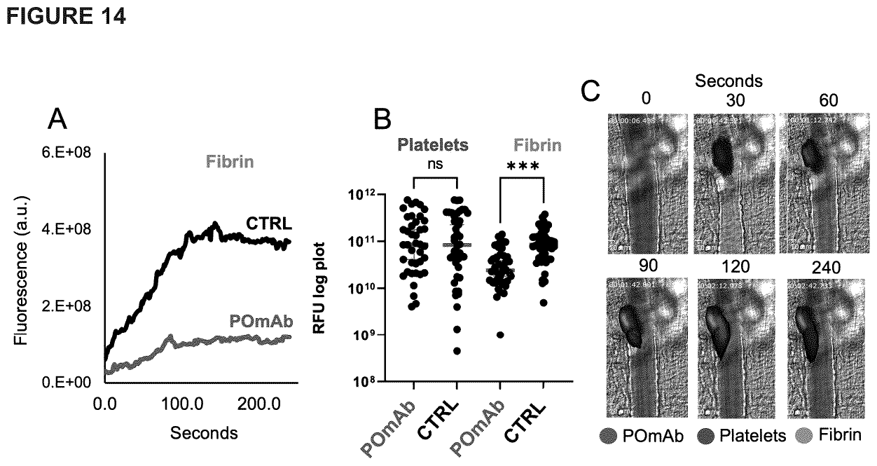

As depicted in FIG. 14, panel A, treatment with 0.5 mg/kg POmAb led to a significant decrease in fibrin accumulation compared to control, without affecting platelet accumulation (FIG. 14, panel B). The images in FIG. 14, panel C illustrate that labeled POmAb prominently accumulated at the injury site. This indicates that POmAb targeted the injury area and exerted an anticoagulant effect by reducing fibrin production.

Having described the invention in detail, it will be apparent that modifications and variations are possible without departing from the scope of the invention defined in the appended claims.

As various changes could be made in the above products and methods without departing from the scope of the invention, it is intended that all matter contained in the above description and shown in the accompanying drawings shall be interpreted as illustrative and not in a limiting sense.

1. A monoclonal antibody against prothrombin, which binds to an open form of prothrombin and decreases conversion of prothrombin to thrombin.

2. The monoclonal antibody of claim 1, which binds to Fragment 1 and Linker 2 of prothrombin.

3. The monoclonal antibody of claim 1, which binds to kringle-1 region of prothrombin.

4. The monoclonal antibody of claims 1-3, which binds to at least one of the following residues E85, S91, Y93, R90 R92 and E111 in the kringle-1 domain of prothrombin; or which binds to at least two of the following residues E85, L88, S91, Y93, R90 R92, Q110, and E111 in the kringle-1 domain of prothrombin.

5. (canceled)

6. The monoclonal antibody of claim 1, wherein the dissociation constant (K_{d}) of the monoclonal antibody is less than about 100 nM.

7. The monoclonal antibody of claim 1, which is a chimeric or humanized antibody.

8. The monoclonal antibody of claim 1, which is a human, humanized or murine antibody comprising complementarity determining region (CDR) sequences of SEQ ID NOs: 3-8.

9.-10. (canceled)

11. The monoclonal antibody of claim 8, comprising sequences at least 90, 95, 98 or 99% identical to the CDR sequences.

12.-14. (canceled)

15. An Fab fragment of the monoclonal antibody of claim 1.

16. An F(ab′)₂ fragment of the monoclonal antibody of claim 1.

17. A conjugate comprising the monoclonal antibody of claim 1 and a label.

18. A single chain antibody fragment (scFv) comprising a sequence of variable heavy chain amino acid sequence of SEQ ID NO: 9 and a sequence of variable light chain amino acid sequence of SEQ ID NO: 10 joined by a 3-20 amino acid linker.

19. An anticoagulant composition, comprising the monoclonal antibody of claim 1 and a physiologically acceptable carrier.

20. A method of treating blood clots in a patient, wherein the method comprises administering the anticoagulant composition of claim 19 to the patient.

21. A method of reducing thrombin generation in a patient, wherein the method comprises administering the anticoagulant composition of claim 19 to the patient.

22. The method of claim 20, wherein treating blood clots or reducing thrombin generation is done prophylactically.

23. The method of claim 20, wherein the patient has or is at risk for deep vein thrombosis (DVT), pulmonary embolism (PE), atrial fibrillation (AFib), myocardial infarction (MI), ischemic stroke, unstable angina, hip or knee replacement surgery, COVID, antiphospholipid syndrome (APS), systemic lupus erythematosus (SLE), or cancer-associated thrombosis (CAT).

24. The method of claims 20-23, wherein the anticoagulant composition is administered intravenously or subcutaneously.

25. (canceled)

26. An anticoagulant composition, comprising the Fab fragment of claim 15 and a physiologically acceptable carrier.

27. An anticoagulant composition, comprising the F(ab′)₂ fragment of claim 16 and a physiologically acceptable carrier.
